(19) United States
(12) Patent Application Publication
(10) Pub. No.: US 2024/0294920 A1
(43) Pub. Date: Sep. 5, 2024

(54) MODULATION OF NOX4 EXPRESSION

(71) Applicant: Ionis Pharmaceuticals, Inc., Carlsbad, CA (US)

(72) Inventors: Jeffrey R. Crosby, Carlsbad, CA (US); Chenguang Zhao, San Diego, CA (US); Alexey Revenko, San Diego, CA (US); Shuling Guo, Carlsbad, CA (US)

(73) Assignee: Ionis Pharmaceuticals, Inc., Carlsbad, CA (US)

(21) Appl. No.: 18/569,084

(22) PCT Filed: Jun. 30, 2022
(86) PCT No.: PCT/US2022/035735

§ 371 Date: Dec. 11, 2023

Related U.S. Application Data
(60) Provisional application No. 63/217,542, filed on Jul. 1, 2021.

(51) Int. Cl. C12N 15/113; A61P 11/00

(52) U.S. Cl. CPC C12N 15/1137; A61P 11/00; C12Y 106/03001; C12N 2310/11; C12N 2310/14; C12N 2310/316; C12N 2310/321; C12N 2310/322; C12N 2310/3231; C12N 2310/3341; C12N 2320/32

(57) ABSTRACT

Provided herein are methods, antisense agents, specific inhibitors, and compositions useful for reducing expression or activity of NADPH oxidase 4 (hereinafter referred to as NOX4) in a subject. Also, provided herein are methods, antisense agents, specific inhibitors, and compositions that can be useful in treating NOX4-related diseases or conditions in a subject. Such methods, antisense agents, specific inhibitors, and compositions can be useful, for example, to treat a pulmonary disease in a subject.

### SEQUENCE LISTING

The present application is being filed along with a Sequence Listing in electronic format. The Sequence Listing is provided as a file entitled BIOL0422WOSEQ_ST25.txt, created on Jun. 21, 2022, which is 612 Kb in size. The information in the electronic format of the sequence listing is incorporated herein by reference in its entirety.

### FIELD

Provided herein are methods, antisense agents, specific inhibitors, and compositions useful for reducing expression or activity of NADPH oxidase 4 (hereinafter referred to as NOX4) in a subject. Also, provided herein are methods, antisense agents, specific inhibitors, and compositions that can be useful in treating NOX4-related diseases or conditions in a subject. Such methods, antisense agents, specific inhibitors, and compositions can be useful, for example, to treat a pulmonary disease in a subject.

### BACKGROUND

Chronic obstructive pulmonary disease (COPD) is a pulmonary condition characterized by chronic obstruction of airflow in the lungs. COPD encompasses a variety of respiratory disorders including emphysema and chronic bronchitis. COPD is characterized by chronic inflammation of the bronchial tubes and is often a result of exposure to toxins such as tobacco smoke. Symptoms of COPD include coughing, wheezing, difficulty breathing, frequent respiratory infections, and coughing with mucus. COPD is a leading cause of morbidity and mortality worldwide (M. Varmaghani et al., East Mediterr Health J. 25, 47-57 (2019); J. L. López-Campos, W. Tan, J. B. Soriano, Respirology. 21, 14-23 (2016)) and poses an increasing public health problem (S. A. Quaderi, J. R. Hurst, Global Health, Epidemiology and Genomics. 3 (2018)).

COPD is a chronic and progressive disease that requires lifelong disease management. The use of bronchodilators can mediate symptoms of COPD (F. Patalano et al., European Respiratory Review. 23, 333-344 (2014)); however, there is currently no cure or treatment available to prevent disease progression (J. Y. Choi, C. K. Rhee, Journal of Clinical Medicine. 9, 3426 (2020), A. Ngkelo, I. M. Adcock, Current Opinion in Pharmacology. 13, 362-369 (2013)).

Pulmonary hypertension (PH) is a complication commonly associated with COPD (A. Chaouat, R. Naeije, E. Weitzenblum, European Respiratory Journal. 32, 1371-1385 (2008)), although PH may be caused by other factors including left-sided heart disease, lung disease, chronic blood clots, and other health conditions (M. D. McGoon, G. C. Kane, Mayo Clin Proc. 84, 191-207 (2009)). In certain circumstances, PH may be considered to be a separate phenomenon than the presumed causal mechanism (M. D. McGoon, G. C. Kane, Mayo Clin Proc. 84, 191-207 (2009)) for example, secondary to idiopathic pulmonary fibrosis (Ruffenach, et al., Respiratory Research, 21, 303 (2020)). Similarly with COPD, individuals with PH often have difficulty breathing and shortness of breath. Further signs and symptoms of PH include chest pressure or pain; edema in ankles, legs, or feet; vascular remodeling; low blood oxygen (hypoxemia); increased blood pressure (hypertension), fatigue, and dizziness. Achieving disease control is difficult in PH (M. Humbert, E. M. T. Lau, CHEST. 156, 1039-1042 (2019)), and currently existing medications do not reverse or prevent progression of the disease (L. D. Harvey, S. Y. Chan, Journal of Clinical Medicine. 6, 43 (2017)).

### SUMMARY

Provided herein are methods, antisense agents, specific inhibitors, and compositions for modulating expression of NOX4-associated with pulmonary diseases or disorders. In certain embodiments, these compositions, compounds, and methods are for modulating the expression of NOX4. In certain embodiments, the NOX4 modulator is a NOX4-specific inhibitor. In certain embodiments, the NOX4-specific inhibitor decreases expression or activity of NOX4. In certain embodiments, NOX4-specific inhibitors include nucleic acids, proteins and small molecules. In certain embodiments, the NOX4-specific inhibitor is a nucleic acid. In certain embodiments, the NOX4-specific inhibitor is an antisense agent. In certain embodiments, the NOX4-specific inhibitor comprises a modified oligonucleotide. In certain embodiments, the modified oligonucleotide can be single stranded or double stranded.

Certain embodiments are directed to compounds useful for inhibiting NOX4, which can be useful for treating a pulmonary disease or disorder. Certain embodiments relate to the novel findings of antisense inhibition of NOX4 resulting in improvement of symptoms or endpoints associated with a pulmonary disease or disorder. Certain embodiments are directed to compounds useful in improving spirometry, lung function, oxygen saturation, or blood pressure, or a combination thereof.

Certain embodiments are described in the numbered embodiments below:
Embodiment 1. A method of treating a pulmonary disease or disorder in a subject having, or at risk of having, a pulmonary disease or disorder comprising administering a NOX4-specific inhibitor to the subject, thereby treating the pulmonary disease or disorder in the subject.
Embodiment 2. The method of embodiment 1, wherein the pulmonary disease or disorder is chronic obstructive pulmonary disease (COPD) or pulmonary hypertension (PH).
Embodiment 3. The method of embodiment 1 or embodiment 2, wherein the administration of the NOX4-specific inhibitor ameliorates at least one symptom of the pulmonary disease or disorder.
Embodiment 4. The method of embodiment 3, wherein the symptom is coughing, wheezing, difficulty breathing, shortness of breath, chest pressure, chest pain, edema in one or both ankles, edema in one or both legs, edema in one or both feet, vascular remodeling, low blood oxygen, increased blood pressure, fatigue, dizziness, frequent respiratory infections, or coughing with mucus.
Embodiment 5. The method of any of embodiments 1-4, wherein administration of the NOX4-specific inhibitor reduces coughing, reduces wheezing, improves breathing, reduces shortness of breath, reduces chest pressure, reduces chest pain, reduces edema in one or both ankles, reduces edema in one or both legs, reduces edema in one or both feet, reduces or reverses vascular remodeling, increases blood oxygen, decreases blood pressure, reduces fatigue, reduces dizziness, reduces the frequency of respiratory infections, reduces coughing with mucus, or a combination thereof.
Embodiment 6. The method of any of embodiments 1-5, wherein administering the NOX4-specific inhibitor improves spirometry, lung function, oxygen saturation, or blood pressure, or a combination thereof.
Embodiment 7. The method of any of embodiments 1-6, wherein administering the NOX4-specific inhibitor reduces bronchial tube inflammation, increases spirometry levels of forced vital capacity, increases spirometry levels of forced expiratory volume, improves lung function, increases oxygen saturation, or decreases blood pressure, or a combination thereof.
Embodiment 8. The method of any of embodiments 1-7, wherein the subject is a human subject.
Embodiment 9. A method of inhibiting expression or activity of NOX4 in a cell comprising contacting the cell with a NOX4-specific inhibitor, thereby inhibiting expression or activity of NOX4 in the cell.
Embodiment 10. The method of embodiment 9, wherein the cell is a human cell.
Embodiment 11. The method of embodiment 9 or embodiment 10, wherein the cell is a lung cell.
Embodiment 12. The method of any of embodiments 9-11, wherein the cell is in a subject.
Embodiment 13. The method of embodiment 12, wherein the subject is human.
Embodiment 14. The method of embodiment 12 or embodiment 13, wherein the subject has, or is at risk of having a pulmonary disease or disorder.
Embodiment 15. The method of embodiment 14, wherein the pulmonary disease or disorder is COPD, or PH.
Embodiment 16. Use of a NOX4-specific inhibitor for the manufacture or preparation of a medicament for treating a pulmonary disease or disorder.
Embodiment 17. Use of a NOX4-specific inhibitor for the treatment of a pulmonary disease or disorder.
Embodiment 18. The use of embodiment 16 or embodiment 17, wherein the pulmonary disease or disorder is COPD or PH.
Embodiment 19. The use of any of embodiments 16-18, wherein the NOX4-specific inhibitor ameliorates at least one symptom of the pulmonary disease or disorder.
Embodiment 20. The use of embodiment 19, wherein the symptom is coughing, wheezing, difficulty breathing, shortness of breath, chest pressure, chest pain, edema in one or both ankles, edema in one or both legs, edema in one or both feet, vascular remodeling, low blood oxygen, increased blood pressure, fatigue, dizziness, frequent respiratory infections, or coughing with mucus Embodiment 21. The use of embodiment 19 or embodiment 20, wherein the NOX4-specific inhibitor reduces coughing, reduces wheezing, improves breathing, reduces shortness of breath, reduces chest pressure, reduces chest pain, reduces edema in one or both ankles, reduces edema in one or both legs, reduces edema in one or both feet, reduces or reverses vascular remodeling, increases blood oxygen, decreases blood pressure, reduces fatigue, reduces dizziness, reduces the frequency of respiratory infections, reduces coughing with mucus, or a combination thereof.
Embodiment 22. The use of any of embodiments 16-21, wherein the NOX4-specific inhibitor improves spirometry, lung function, oxygen saturation, or blood pressure, or a combination thereof.
Embodiment 23. The use of any of embodiments 16-22, wherein the NOX4-specific inhibitor reduces bronchial tube inflammation, increases spirometry levels of forced vital capacity, increases spirometry levels of forced expiratory volume, improves lung function, increases oxygen saturation, or decreases blood pressure, or a combination thereof.
Embodiment 24. The method or use of any of embodiments 1-23, wherein the NOX4-specific inhibitor is an antisense agent, a polypeptide, an antibody, or a small molecule.
Embodiment 25. The method or use of any of embodiments 1-24, wherein the NOX4-specific inhibitor is an antisense agent comprising a modified oligonucleotide, wherein the modified oligonucleotide has a nucleobase sequence complementary to any one of SEQ ID NOs: 1-5.
Embodiment 26. The method or use of embodiment 25, wherein the nucleobase sequence of the modified oligonucleotide is complementary to SEQ ID NO: 3 or SEQ ID NO: 4.
Embodiment 27. The method or use of embodiment 26, wherein the nucleobase sequence of the modified oligonucleotide is at least 90% complementary to the nucleobase sequence of an equal length portion of SEQ ID NO: 3 or SEQ ID NO: 4.
Embodiment 28. The method or use of embodiment 26, wherein the nucleobase sequence of the modified oligonucleotide is at least 95% complementary to the nucleobase sequence of an equal length portion of SEQ ID NO: 3 or SEQ ID NO: 4.
Embodiment 29. The method or use of embodiment 28, wherein the nucleobase sequence of the modified oligonucleotide is 100% complementary to the nucleobase sequence of an equal length portion of SEQ ID NO: 3 or SEQ ID NO: 4.
Embodiment 30. The method or use of any of embodiments 24-29, wherein the antisense agent is single-stranded.
Embodiment 31. The method or use of any of embodiments 24-29, wherein the antisense agent is double-stranded.
Embodiment 32. The method or use of any of embodiments 25-29, wherein the modified oligonucleotide consists of 12 to 30 linked nucleosides.
Embodiment 33. The method or use of any of embodiments 25-32, wherein at least nucleoside of the modified oligonucleotide comprises a modified nucleobase.
Embodiment 34. The method or use of embodiment 33, wherein the modified nucleobase is a 5-methylcytosine.
Embodiment 35. The method or use of any of embodiments 25-34, wherein at least one nucleoside of the modified oligonucleotide comprises a modified sugar moiety.
Embodiment 36. The method or use of embodiment 35, wherein the modified sugar moiety is a bicyclic sugar moiety.
Embodiment 37. The method or use of embodiment 36, wherein the modified sugar comprises a 4′-CH(CH₃)—O-2′ bridge or a 4′-(CH₂)ₙ—O-2′ bridge, wherein n is 1 or 2.
Embodiment 38. The method or use of embodiment 37, wherein the modified sugar moiety comprises a non-bicyclic modified sugar moiety.
Embodiment 39. The method or use of embodiment 38, wherein the non-bicyclic sugar moiety is a 2′-F, 2′-OMe, or 2′-MOE sugar moiety.
Embodiment 40. The method or use of any of embodiments 25-39, wherein the modified oligonucleotide has:
   a gap segment consisting of linked 2′-deoxynucleosides;
   a 5′ wing segment consisting of linked nucleosides;
   a 3′ wing segment consisting linked nucleosides;
   wherein the gap segment is positioned immediately adjacent to and between the 5′ wing segment and the 3′ wing segment and wherein each nucleoside of each wing segment comprises a modified sugar moiety.
Embodiment 41. The method or use of any of embodiments 25-39, wherein the modified oligonucleotide has
   a sugar motif comprising:
   a 5′-region consisting of 1-6 linked 5′-region nucleosides;
      a central region consisting of 6-10 linked central region nucleosides; and
      a 3′-region consisting of 1-6 linked 3′-region nucleosides;
   wherein the 3′-most nucleoside of the 5′-region and the 5′-most nucleoside of the 3′-region comprise modified sugar moieties, and
   each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprise a 2′-substituted sugar moiety.
Embodiment 42. The method or use of embodiment 41, wherein the central region consists of 6-10 nucleosides comprising a 2′-β-D-deoxyribosyl sugar moiety.
Embodiment 43. The method or use of any one of embodiments 25-42, wherein at least one internucleoside linkage of the modified oligonucleotide is a modified internucleoside linkage.
Embodiment 44. The method or use of embodiment 43, wherein the at least one modified internucleoside linkage is a phosphorothioate internucleoside linkage.
Embodiment 45. The method or use of embodiment 44, wherein each internucleoside linkage is a phosphorothioate internucleoside linkage.
Embodiment 46. The method or use of embodiment 44, wherein each internucleoside linkage is independently selected from a phosphodiester internucleoside linkage and a phosphorothioate internucleoside linkage.
Embodiment 47. The method or use of any of embodiments 24-46, wherein the antisense agent comprises a conjugate group.
Embodiment 48. The method or use of any of embodiments 1-47, wherein the NOX4-specific inhibitor is an RNase H agent capable of reducing the amount of NOX4 nucleic acid through the activation of RNase H.
Embodiment 49. The method or use of any of embodiments 1-47, wherein the NOX4-specific inhibitor is an RNAi agent capable of reducing the amount of NOX4 nucleic acid through the activation of RISC/Ago2.
Embodiment 50. The method or use of any of embodiments 1-47, wherein the NOX4-specific inhibitor is a steric-blocking agent capable of directly binding to a target nucleic acid, thereby blocking the interaction of the NOX4 nucleic acid with other nucleic acids or proteins.
Embodiment 51. The method of any of embodiments 1-8 or 16-50, wherein a therapeutic amount of the NOX4-specific inhibitor is administered to the subject.
Embodiment 52. The method of any of embodiments 1-8 or 16-51, wherein the NOX4-specific inhibitor is administered by aerosolized delivery.
Embodiment 53. The method of any of embodiments 1-8 or 16-51, wherein the NOX4-specific inhibitor is administered via a nebulizer or inhaler.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ISIS/IONIS number (ISIS/ION #) indicate a combination of nucleobase sequence, chemical modification, and motif.

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings: “2′-deoxynucleoside” means a nucleoside comprising 2′-H(H) furanosyl sugar moiety, as found in naturally occurring deoxyribonucleic acids (DNA). In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

“2′-MOE” means a 2′-OCH₂CH₂OCH₃ group in place of the 2′-OH group of a furanosyl sugar moiety. A “2′-MOE sugar moiety” or a “2′-MOE modified sugar moiety” means a sugar moiety with a 2′-OCH₂CH₂OCH₃ group in place of the 2′-OH group of a furanosyl sugar moiety. Unless otherwise indicated, a 2′-MOE sugar moiety is in the β-D-ribosyl configuration. “MOE” means O-methoxyethyl. “2′-MOE nucleoside” (also 2′-O-methoxyethyl nucleoside) means a nucleoside comprising a 2′-MOE sugar moiety.

“2′-OMe” means a 2′-OCH₃ group in place of the 2′-OH group of a furanosyl sugar moiety. A″2′-O-methyl sugar moiety” or “2′-OMe sugar moiety” or a “2′-OMe modified sugar moiety” means a sugar moiety with a 2′-OCH₃ group in place of the 2′-OH group of a furanosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “2′-F” means a 2′-fluoro group in place of the 2′-OH group of a ribosyl sugar moiety. A “2′-F sugar moiety” or “2′-fluororibosyl sugar moiety” means a sugar moiety with a 2′-F group in place of the 2′-OH group of a ribosyl sugar moiety. Unless otherwise indicated, a 2′-F has the β-D ribosyl stereochemical configuration.

As used herein, “2′-F nucleoside” means a nucleoside comprising a 2′-F sugar moiety.
“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.
“3′ target site” refers to the nucleotide of a target nucleic acid which is complementary to the 3′-most nucleotide of a particular compound.
“5′ target site” refers to the nucleotide of a target nucleic acid which is complementary to the 5′-most nucleotide of a particular compound.
“5-methylcytosine” means a cytosine with a methyl group attached to the 5 position. A 5-methylcytosine is a modified nucleobase.

“About” means within +10% of a value. For example, if it is stated, “the compounds affected about 70% inhibition of NOX4”, it is implied that NOX4 levels are inhibited within a range of 60% and 80%.

As used herein, “administration” or “administering” means providing a pharmaceutical agent or composition to an animal.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Ameliorate” refers to an improving or lessening of at least one indicator, sign, hallmark, or symptom of an associated disease, disorder, or condition relative to the same indicator, sign, hallmark, or symptom in the absence of the treatment. In certain embodiments, amelioration includes a delay or slowing in the progression or severity of one or more indicators of a condition or disease. The progression or severity of indicators may be determined by subjective or objective measures, which are known to those skilled in the art. In certain embodiments, amelioration is the reduction in the severity or frequency of a symptom or hallmark or the delayed onset or slowing of progression in the severity or frequency of a symptom or hallmark. In certain embodiments, the symptom or hallmark is one or more of coughing, wheezing, difficulty breathing, shortness of breath, chest pressure, chest pain, edema in one or both ankles, edema in one or both legs, or edema in one or both feet, vascular remodeling, low blood oxygen, increased blood pressure, fatigue, dizziness, frequent respiratory infections, or coughing with mucus.

“Antisense activity” means any detectable and/or measurable change in an amount of a target nucleic acid, or protein encoded by such target nucleic acid, attributable to the hybridization of an antisense compound to its target nucleic acid. In certain embodiments, antisense activity is a decrease in the amount of a target nucleic acid, or protein encoded by such target nucleic acid, compared to the amount of target nucleic acid, or protein encoded by such target nucleic acid, in the absence of the antisense compound. In certain embodiments, the change is detectable in a cell that has been contacted with the antisense compound or a cell lysate thereof. In certain embodiments, the change is detectable in a biological sample obtained from a subject to whom the antisense compound has been administered. Non-limiting examples of biological samples include a liver biopsy sample, a blood sample, a plasma/serum sample, a saliva sample, a urine sample, a lung biopsy sample, a bronchial brushings sample, a bronchoalveolar lavage sample, a saliva sample, a sputum sample, and a breath condensate sample.

“Antisense agent” means an antisense compound and optionally one or more additional features, such as a sense compound. An antisense agent includes, but is not limited to, an RNAi agent and an RNase H agent.

“Antisense compound” means an oligonucleotide, such as an antisense oligonucleotide, and optionally one or more additional features, such as a conjugate group

“Sense compound” means a sense oligonucleotide and optionally one or more additional features, such as a conjugate group.

“Antisense inhibition” means reduction of target nucleic acid levels in the presence of an antisense agent or antisense compound comprising an oligonucleotide complementary to a target nucleic acid compared to target nucleic acid levels in the absence of the antisense compound.

“Antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

“Sense oligonucleotide” means an oligonucleotide, including the oligonucleotide portion of a sense compound, that is capable of hybridizing to an antisense oligonucleotide.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl sugar moiety is a ribosyl sugar moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a bicyclic furanosyl sugar moiety comprising a bridge connecting the 4′-carbon and the 2′-carbon, wherein the bridge has the formula: 4′-CH(CH₃)—O-2′. As used herein, “constrained ethyl nucleoside” or “cEt nucleoside” means:

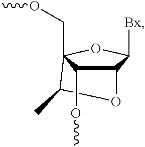

wherein Bx is a nucleobase

“Constrained ethyl” or “cEt” or “cEt sugar moiety” means the sugar moiety of a cEt nucleoside.

“Chemical modification” in a compound describes the substitutions or changes through chemical reaction, of any of the units in the compound. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Complementary” in reference to an oligonucleotide means that at least 70% of the nucleobases of the oligonucleotide and the nucleobases of another nucleic acid or one or more regions thereof are capable of hydrogen bonding with one another when the nucleobase sequence of the oligonucleotide and the other nucleic acid are aligned in opposing directions. “Complementary region” in reference to a region of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions. Complementary nucleobases mean nucleobases that are capable of forming hydrogen bonds with one another. Complementary nucleobase pairs include adenine (A) and thymine (T), adenine (A) and uracil (U), cytosine (C) and guanine (G), 5-methylcytosine (mC) and guanine (G). Certain modified nucleobases that pair with natural nucleobases or with other modified nucleobases are known in the art and are not considered complementary nucleobases as defined herein unless indicated otherwise. For example, inosine can pair, but is not considered complementary, with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide. 5-methylcytosine“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker. A conjugate moiety modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” mean chemical modifications or chemical substituents that are different from one another, including absence of modifications. Thus, for example, a 2′-MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe sugar moiety and an unmodified adenine nucleobase and a nucleoside comprising a 2′-OMe sugar moiety and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, injections, or inhalations. In certain embodiments, a dose may be aerosolized. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in a subject. In other embodiments, the compound or pharmaceutical agent is administered by infusion over an extended period of time or continuously. Doses may be stated as the amount of pharmaceutical agent per hour, day, week or month.

“Double-stranded” in reference to an antisense agent means the antisense agent has two oligonucleotides that are sufficiently complementary to each other to form a duplex. “Double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

“Efficacy” means the ability to produce a desired effect.

“Expression” includes all the functions by which a gene's coded information is converted into structures present and operating in a cell. Such structures include, but are not limited to the products of transcription and translation.

“Gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications. “Hybridization” means annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Inhaled administration” means administration within the respiratory tract by inhaling orally or nasally for local or systemic effect. Inhaled administration may involve aerosolization and/or dispersal by a nebulizer or inhaler.

“Inhibiting or reducing the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Linked nucleosides” means adjacent nucleosides linked together by an internucleoside linkage.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating NOX4 can mean to increase or decrease the level of NOX4 in a cell, tissue, organ or organism. A “modulator” effects the change in the cell, tissue, organ or organism. For example, a compound can be a modulator of NOX4 that decreases the amount of NOX4 in a cell, tissue, organ or organism.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“NOX4” means NADPH oxidase 4 and refers to any NOX4 nucleic acid or NOX4 protein. In certain embodiments, NOX4 includes a DNA sequence encoding NOX4, an RNA sequence transcribed from DNA encoding NOX4 (including genomic DNA comprising introns and exons), or a NOX4 protein. The target may be referred to in either upper or lower case.

“NOX4-specific inhibitor” refers to any agent capable of specifically reducing NOX4 RNA or NOX4 protein in a cell relative to a cell that is not exposed to the agent. NOX4-specific inhibitors include nucleic acids (including antisense compounds), peptides, antibodies, small molecules, and other agents capable of inhibiting the expression or activity of NOX4.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is a modified nucleobase. A universal base is a modified nucleobase that can pair with any one of the five unmodified nucleobases. “Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

“Oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

“Oligomeric duplex” means a duplex formed by two oligomeric compounds having complementary nucleobase sequences.

“Oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. “Parent oligonucleotide” means an oligonucleotide having a nucleobase sequence that is used as the basis of design for more oligonucleotides of similar sequence but with different lengths, motifs, and/or chemistries. The newly designed oligonucleotides may have the same or overlapping sequence as the parent oligonucleotide.

“Pharmaceutically acceptable carrier or diluent” means any substance suitable for use in administering to a subject. For example, a pharmaceutically acceptable carrier can be a sterile aqueous solution, such as PBS or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical agent” means a compound that provides a therapeutic benefit when administered to a subject.

“Pharmaceutical composition” means a mixture of substances suitable for administering to a subject.

For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prodrug” means a compound in a form outside the body which, when administered to a subject, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Pulmonary disease” or “Pulmonary disorder” means a condition of the lung that reduces lung function, such as inhalation and/or exhalation. Pulmonary diseases and disorders can be caused by a genetic mutation, an infection, an injury, exposure to an environmental toxin, or a combination thereof.

“Reduce” means to bring down to a smaller extent, size, amount, or number. In certain embodiments, NOX4 (RNA or protein) is reduced in a cell or individual that is contacted or treated with a NOX4-specific inhibitor, respectively, relative to a cell or individual that is not contacted or treated with a NOX4-specific inhibitor, respectively.

“RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics.

RNAi agents may comprise conjugate groups and/or terminal groups. In certain embodiments, an RNAi agent modulates the amount and/or activity, of a target nucleic acid. The term RNAi agent excludes antisense agents that act through RNase H.

“RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“Segments” are defined as smaller or sub-portions of regions within a nucleic acid.

“Single-stranded” in reference to an antisense agent means the antisense agent has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites,” are defined as unique nucleobase positions within a target nucleic acid.

“Specifically hybridizable” and “specific hybridization” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” a target nucleic acid means to reduce or block expression of the target nucleic acid while exhibiting fewer, minimal, or no effects on non-target nucleic acids reduction and does not necessarily indicate a total elimination of the target nucleic acid's expression.

“Subject” means a human or non-human subject selected for treatment or therapy.

“Sugar moiety” means an unmodified sugar moiety or a modified sugar moiety. “Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

“Target gene” refers to a gene encoding a target.

“Targeting” and “targeted” means specific hybridization of an antisense agent, antisense compound that, or oligonucleotide to a target nucleic acid in order to induce a desired effect.

“Target nucleic acid,” “target RNA,” “target RNA transcript” and “nucleic acid target” all mean a nucleic acid capable of being targeted by compounds described herein. Target RNA means an RNA transcript and includes pre-mRNA and mature mRNA unless otherwise specified.

“Target region” means a portion of a target nucleic acid to which one or more compounds is targeted.

“Target segment” means the sequence of nucleotides of a target nucleic acid to which a compound described herein is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to a subject.

“Treat” refers to administering a compound or pharmaceutical composition to a subject in order to effect an alteration or improvement of a disease, disorder, or condition in the subject. In certain embodiments, treating a subject improves a symptom relative to the same symptom in the absence of the treatment. In certain embodiments, treatment reduces the severity or frequency of a symptom, or delays the onset of a symptom, slows the progression of a symptom, or slows the severity or frequency of a symptom.

### Certain Embodiments

Certain embodiments provide methods, NOX4-specific inhibitors, and compositions for treating a pulmonary disease condition, or a symptom thereof, in a subject by administering the compound or composition to the subject. Inhibition of NOX4 can lead to a decrease of NOX4 level or expression in order to treat a pulmonary disease or disorder, or a symptom thereof. In certain embodiments, NOX4-specific inhibitors are antisense agents, single-stranded antisense agents, double-stranded antisense agents, RNAi agents, RNase H agents, double-stranded siRNA, single-stranded RNAi (ssRNAi), microRNA, antisense compounds, oligonucleotides, peptides, antibodies, small molecules, and other agents capable of inhibiting the expression or activity of NOX4. In certain embodiments, the subject is human. In certain embodiments, the antisense agent or RNAi agent comprises ribonucleotides and is double-stranded. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide.

In any of the foregoing embodiments, the modified oligonucleotide consists of 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 20, 18 to 22, 18 to 24, 18 to 30, 18 to 50, 19 to 22, 19 to 30, 19 to 50, or 20 to 30 linked nucleosides.

In certain embodiments, at least one internucleoside linkage of said modified oligonucleotide is a modified internucleoside linkage. In certain embodiments, at least one internucleoside linkage is a phosphorothioate internucleoside linkage. In certain embodiments, the internucleoside linkages are phosphorothioate linkages and phosphate ester linkages.

In certain embodiments, any of the foregoing oligonucleotides comprises at least one modified sugar. In certain embodiments, at least one modified sugar comprises a 2′-O-methoxyethyl group. In certain embodiments, at least one modified sugar is a bicyclic sugar, such as a 4′-CH(CH₃)—O-2′ group, a 4′-CH₂—O-2′ group, or a 4′-(CH₂)₂—O-2′ group. In certain embodiments, at least one modified sugar comprises a 2′-F group or a 2′-OMe group.

In certain embodiments, at least one nucleoside of said modified oligonucleotide comprises a modified nucleobase. In certain embodiments, the modified nucleobase is a 5-methylcytosine.

In certain embodiments, a compound or composition comprises a modified oligonucleotide comprising: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, at least one internucleoside linkage is a phosphorothioate linkage. In certain embodiments, and at least one cytosine is a 5-methylcytosine.

In certain embodiments, the compounds or compositions disclosed herein further comprise a pharmaceutically acceptable carrier or diluent.

In certain embodiments, NOX4-specific inhibitors can be used in methods of inhibiting expression of NOX4 in a cell. In certain embodiments, NOX4-specific inhibitors can be used in methods of treating a pulmonary disease or disorder including, but not limited to, chronic obstructive pulmonary disease or pulmonary hypertension.

### Certain Indications

Certain embodiments provided herein relate to methods of inhibiting NOX4 expression or activity, which can be useful for treating a disease associated with NOX4 in a subject, such as COPD or PH, by administration of a NOX4-specific inhibitor.

In certain embodiments, a method of inhibiting expression or activity of NOX4 in a cell comprises contacting the cell with a compound or composition comprising a NOX4-specific inhibitor, thereby inhibiting expression or activity of NOX4 in the cell. In certain embodiments, the cell is a lung cell. In certain embodiments, the cell is in the lung. In certain embodiments, the cell is in the lung of a subject who has, or is at risk of having a disease, disorder, condition, symptom, or physiological marker associated with a pulmonary disease or disorder. In certain embodiments, the pulmonary disease or disorder is chronic obstructive pulmonary disease or pulmonary hypertension. In certain embodiments, the NOX4-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the NOX4. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the modified oligonucleotide consists of 16 to 20 linked nucleosides. In certain embodiments, the modified oligonucleotide consists of 18 to 20 linked nucleosides. In certain embodiments, the antisense agent comprises a single-stranded modified oligonucleotide and is a single-stranded antisense agent. In certain embodiments, the antisense agent comprises a double-stranded modified oligonucleotide and is a double-stranded antisense agent. In certain embodiments, the double-stranded antisense agent comprises an antisense compound and a sense compound. In certain embodiments, the antisense agent is a double-stranded siRNA targeted to NOX4.

In certain embodiments, a method of treating one or more diseases, disorders, conditions, symptoms or physiological markers associated with NOX4 comprises administering to the subject a NOX4-specific inhibitor. In certain embodiments, a method of treating a disease, disorder, condition, symptom, or physiological marker associated with a pulmonary disease or disorder in a subject comprises administering to the subject a NOX4-specific inhibitor, thereby treating the disease. In certain embodiments, the subject is identified as having, or at risk of having, the disease, disorder, condition, symptom or physiological marker.

In certain embodiments, the pulmonary disease or disorder is COPD or PH. In certain embodiments, the NOX4-specific inhibitor is administered to the subject by aerosolized delivery. In certain embodiments, the NOX4-specific inhibitor is administered via a nebulizer or inhaler. In certain embodiments, the NOX4-specific inhibitor is administered via a nebulizer or inhaler, wherein the NOX4-inhibitor is administered as a mixture comprising, or in the presence of, a pharmaceutically acceptable excipient. In certain embodiments, the subject is human. In certain embodiments, the NOX4-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the NOX4. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the antisense agent comprises a single-stranded modified oligonucleotide and is a single-stranded antisense agent. In certain embodiments, the antisense agent comprises a double-stranded modified oligonucleotide and is a double-stranded antisense agent. In certain embodiments, the double-stranded antisense agent comprises an antisense compound and a sense compound. In certain embodiments, the antisense agent is a double-stranded siRNA targeted to NOX4.

In certain embodiments, a method of improving spirometry, lung function, oxygen saturation, or blood pressure, or a combination thereof, in a subject comprises administering to the subject a NOX4-specific inhibitor. In certain embodiments, spirometry, lung function, oxygen saturation, or blood pressure, or a combination thereof, is improved in a subject that is administered a NOX4-specific inhibitor, relative to spirometry, lung function, oxygen saturation, or blood pressure, or a combination thereof in the subject before administration. In certain embodiments, spirometry, lung function, oxygen saturation, or blood pressure, or a combination thereof, is reduced in a subject that is administered a NOX4-specific inhibitor, relative to spirometry, lung function, oxygen saturation, or blood pressure, or a combination thereof in a control subject that does not receive the NOX4-specific inhibitor. In certain embodiments, the subject is identified as having, or at risk of having a disease, disorder, condition, symptom, or physiological marker associated with a pulmonary disease or disorder. In certain embodiments, the pulmonary disease or disorder is COPD or PH. In certain embodiments, the NOX4-specific inhibitor is administered to the subject by aerosolized delivery. In certain embodiments, the NOX4-specific inhibitor is administered via a nebulizer or inhaler. In certain embodiments, the NOX4-specific inhibitor is administered via a nebulizer or inhaler, wherein the NOX4-inhibitor is administered as a mixture comprising, or in the presence of, a pharmaceutically acceptable excipient. In certain embodiments, the subject is human. In certain embodiments, the NOX4-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the NOX4. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the modified oligonucleotide consists of 16 to 20 linked nucleosides. In certain embodiments, the modified oligonucleotide consists of 18 to 20 linked nucleosides. In certain embodiments, the antisense agent comprises a single-stranded modified oligonucleotide and is a single-stranded antisense agent. In certain embodiments, the antisense agent comprises a double-stranded modified oligonucleotide and is a double-stranded antisense agent. In certain embodiments, the double-stranded antisense agent comprises an antisense compound and a sense compound. In certain embodiments, the antisense agent is a double-stranded siRNA targeted to NOX4.

Certain embodiments are drawn to compounds and compositions described herein for use in therapy. Certain embodiments are drawn to a compound or composition comprising a NOX4-specific inhibitor for use in treating one or more diseases, disorders, conditions, symptoms or physiological markers associated with NOX4. Certain embodiments are drawn to a compound or composition for use in treating a pulmonary disease or disorder, or a symptom or physiological marker thereof. In certain embodiments, the pulmonary disease or disorder is COPD or PH. In certain embodiments, the NOX4-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the NOX4. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the modified oligonucleotide consists of 16 to 20 linked nucleosides. In certain embodiments, the modified oligonucleotide consists of 18 to 20 linked nucleosides. In certain embodiments, the antisense agent comprises a single-stranded modified oligonucleotide and is a single-stranded antisense agent. In certain embodiments, the antisense agent comprises a double-stranded modified oligonucleotide and is a double-stranded antisense agent. In certain embodiments, the double-stranded antisense agent comprises an antisense compound and a sense compound. In certain embodiments, the antisense agent is a double-stranded siRNA targeted to NOX4.

Certain embodiments are drawn to a NOX4-specific inhibitor or composition comprising a NOX4-specific inhibitor for use in improving spirometry, lung function, oxygen saturation, or blood pressure, or a combination thereof, in a subject. In certain embodiments, spirometry, lung function, oxygen saturation, or blood pressure, or a combination thereof, is improved in a subject that is administered a NOX4-specific inhibitor, relative to spirometry, lung function, oxygen saturation, or blood pressure, or a combination thereof in the subject before administration. In certain embodiments, spirometry, lung function, oxygen saturation, or blood pressure, or a combination thereof, is reduced in a subject that is administered a NOX4-specific inhibitor, relative to spirometry, lung function, oxygen saturation, or blood pressure, or a combination thereof in a control subject that does not receive the NOX4-specific inhibitor. In certain embodiments, the compound or composition is provided for use in improving spirometry in the subject. In certain embodiments, the NOX4-specific inhibitor or composition is provided for use in improving lung function in the subject. In certain embodiments, the compound or composition is provided for use in improving oxygen saturation in the subject. In certain embodiments, the NOX4-specific inhibitor or composition is provided for use in improving blood pressure in the subject. In certain embodiments, the subject is identified as having, or at risk of having a disease, disorder, condition, symptom, or physiological marker associated with a pulmonary disease or disorder. In certain embodiments, the pulmonary disease or disorder is COPD or PH. In certain embodiments, the subject is a human subject. In certain embodiments, the NOX4-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the NOX4. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the modified oligonucleotide consists of 16 to 20 linked nucleosides. In certain embodiments, the modified oligonucleotide consists of 18 to 20 linked nucleosides. In certain embodiments, the antisense agent comprises a single-stranded modified oligonucleotide and is a single-stranded antisense agent. In certain embodiments, the antisense agent comprises a double-stranded modified oligonucleotide and is a double-stranded antisense agent. In certain embodiments, the double-stranded antisense agent comprises an antisense compound and a sense compound. In certain embodiments, the antisense agent is a double-stranded siRNA targeted to NOX4.

Certain embodiments are drawn to use of NOX4-specific inhibitors or compositions described herein for the manufacture or preparation of a medicament for therapy. Certain embodiments are drawn to the use of a NOX4-specific inhibitor or composition as described herein in the manufacture or preparation of a medicament for treating one or more diseases, disorders, conditions, symptoms or physiological markers associated with NOX4. In certain embodiments, the compound or composition as described herein is used in the manufacture or preparation of a medicament for treating a pulmonary disease or disorder, or a symptom or physiological marker thereof. In certain embodiments, the pulmonary disease or disorder is COPD or PH. In certain embodiments, the NOX4-specific inhibitor is an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the NOX4. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the modified oligonucleotide consists of 16 to 20 linked nucleosides. In certain embodiments, the modified oligonucleotide consists of 18 to 20 linked nucleosides. In certain embodiments, the antisense agent comprises a single-stranded modified oligonucleotide and is a single-stranded antisense agent. In certain embodiments, the antisense agent comprises a double-stranded modified oligonucleotide and is a double-stranded antisense agent. In certain embodiments, the double-stranded antisense agent comprises an antisense compound and a sense compound. In certain embodiments, the antisense agent is a double-stranded siRNA targeted to NOX4.

Certain embodiments are drawn to the use of a NOX4-specific inhibitor or composition for the manufacture or preparation of a medicament for improving spirometry, lung function, oxygen saturation, or blood pressure, or a combination thereof, in a subject having or at risk of having a pulmonary disease or disorder. Certain embodiments are drawn to use of a NOX4-specific inhibitor or composition in the manufacture or preparation of a medicament for improving spirometry in the subject. Certain embodiments are drawn to use of a NOX4-specific inhibitor in the manufacture or preparation of a medicament for improving lung function in the subject. Certain embodiments are drawn to use of a NOX4-specific inhibitor in the manufacture or preparation of a medicament for improving oxygen saturation in the subject. Certain embodiments are drawn to use of a NOX4-specific inhibitor in the manufacture or preparation of a medicament for improving blood pressure in the subject. In certain embodiments, the compound or composition comprises an antisense agent, single-stranded antisense agent, double-stranded antisense agent, RNAi agent, RNase H agent, double-stranded siRNA, single-stranded RNAi (ssRNAi), microRNA, antisense compound, peptide, antibody, small molecule or other agent capable of inhibiting the expression or activity of the NOX4. In certain embodiments, the antisense agent, single-stranded antisense agent, double-stranded antisense agent, RNAi agent, RNase H agent, double-stranded siRNA, single-stranded RNAi (ssRNAi), microRNA, or antisense compound comprises a modified oligonucleotide consisting of 8 to 80 linked nucleosides. In certain embodiments, the modified oligonucleotide consists of 10 to 30 linked nucleosides. In certain embodiments, the modified oligonucleotide consists of 16 to 20 linked nucleosides. In certain embodiments, the modified oligonucleotide consists of 18 to 20 linked nucleosides. In certain embodiments, the antisense agent comprises a single-stranded modified oligonucleotide and is a single-stranded antisense agent. In certain embodiments, the antisense agent comprises a double-stranded modified oligonucleotide and is a double-stranded antisense agent. In certain embodiments, the double-stranded antisense agent comprises an antisense compound and a sense compound. In certain embodiments, the antisense agent is a double-stranded siRNA targeted to NOX4.

In any of the foregoing methods or uses, the antisense agent can comprise an antisense compound targeted to NOX4. In certain embodiments, the antisense compound comprises an oligonucleotide, for example an oligonucleotide consisting of 8 to 80 linked nucleosides, 10 to 30 linked nucleosides, 12 to 30 linked nucleosides, 16 to 20 linked nucleosides, 18 to 20 linked nucleosides, or 20 linked nucleosides. In certain embodiments, the oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′-O-methoxyethyl, and the modified nucleobase is a 5-methylcytosine. In certain embodiments, the modified oligonucleotide comprises a gap segment consisting of linked deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar. In certain embodiments, the modified oligonucleotide comprises a gap segment consisting of linked deoxynucleosides; a 5′-region consisting of linked nucleosides; and a 3′-region consisting of linked nucleosides, wherein the gap segment is positioned immediately adjacent to and between the 5′-region and the 3′-region, wherein the 3′-most nucleoside of the 5′-region and the 5′-most nucleoside of the 3′-region comprise modified sugar moieties.

In certain embodiments, the antisense agent is single-stranded. In certain embodiments, the antisense agent is double-stranded. In certain embodiments, the modified oligonucleotide consists of 12 to 30 linked nucleosides. In certain embodiments, compositions disclosed herein comprise an antisense agent described herein and a pharmaceutically acceptable carrier or diluent.

In any of the foregoing methods or uses, the compound or composition may comprise or consists of a modified oligonucleotide 12 to 30 linked nucleosides in length, wherein the modified oligonucleotide comprises:
a gap segment consisting of linked 2′-deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

In any of the foregoing methods or uses, the compound or composition may comprise or consists of a modified oligonucleotide 12 to 30 linked nucleosides in length, wherein the modified oligonucleotide comprises:
a central region consisting of 6-10 linked central region nucleosides;
a 5′-region consisting of 1-6 linked 5′-region nucleosides; and
a 3′-region consisting of 1-6 linked 3′-region nucleosides;
   wherein the 3′-most nucleoside of the 5′-region and the 5′-most nucleoside of the 3′-region comprise modified sugar moieties, and
   each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprise a 2′-substituted sugar moiety. In certain embodiments, each of the central region nucleosides comprises a 2′-β-D-deoxyribosyl sugar moiety. In certain embodiments, each of the 5′-region nucleosides comprises a modified sugar moiety. In certain embodiments, each of the 3′-region nucleosides comprises a modified sugar moiety. In certain embodiments, each of the 5′-region nucleosides and each of the 3′-region nucleosides comprises a modified sugar moiety. In certain embodiments, one or more of the 5′-region nucleosides comprises a 2′-β-D-deoxyribosyl sugar moiety. In certain embodiments, one or more of the 5′-region nucleosides comprises a 2′-β-D-deoxyribosyl sugar moiety.

In any of the foregoing methods or uses, the compound or composition can be administered via inhalation. Certain embodiments provide compounds or compositions suitable for aerosolization and/or dispersal by a nebulizer or inhaler. In certain embodiments, the compound or composition is a solid comprising particles of compounds that are of respirable size. A solid particulate composition can optionally contain a dispersant which serves to facilitate the formation of an aerosol, e.g., lactose. Solid pharmaceutical compositions comprising an oligonucleotide can also be aerosolized using any solid particulate medicament aerosol generator known in the art, e.g., a dry powder inhaler. In certain embodiments, the powder employed in the inhaler consists of the compound comprising the active compound or of a powder blend comprising the active compound, a suitable powder diluent, and an optional surfactant. In certain embodiments, the pharmaceutical composition is a liquid. In certain such embodiments, the liquid is administered as an aerosol that is produced by any suitable means, such as with a nebulizer or inhaler. See, e.g., U.S. Pat. No. 4,501,729. In certain embodiments, the nebulizer is a device for producing a spray of liquid. Nebulizers are devices that transform solutions or suspensions into an aerosol mist and are well known in the art. Suitable nebulizers include jet nebulizers, ultrasonic nebulizers, electronic mesh nebulizers, and vibrating mesh nebulizers. In certain embodiments, the nebulizer is activated manually by squeezing a flexible bottle that contains the pharmaceutical composition. In certain embodiments, the aerosol is produced by a metered dose inhaler, which typically contains a suspension or solution formulation of the active compound in a liquefied propellant. Pharmaceutical compositions suitable for aerosolization can comprise propellants, surfactants, co-solvents, dispersants, preservatives, and/or other additives or excipients. Examples of pharmaceutically acceptable excipients have been described previously (for example, Pilcer and Amighi, Int. J. Pharmaceutics, 2010, 392, 1-19).

### Certain Compounds

In certain embodiments, antisense agents described herein comprise antisense compounds. In certain embodiments, the antisense compound comprises a modified oligonucleotide. In certain embodiments, the modified oligonucleotide has a nucleobase sequence complementary to that of a target nucleic acid.

In certain embodiments, an antisense agent described herein comprises or consists of a modified oligonucleotide. In certain embodiments, the modified oligonucleotide has a nucleobase sequence complementary to that of a target nucleic acid.

In certain embodiments, an antisense agent is single-stranded. In certain embodiments, a single-stranded antisense agent comprises or consists of an antisense compound. In certain embodiments, such an antisense compound comprises or consists of an oligonucleotide. In certain embodiments, the oligonucleotide is an antisense oligonucleotide. In certain embodiments, the oligonucleotide is modified. In certain embodiments, the oligonucleotide of a single-stranded antisense agent or antisense compound comprises a self-complementary nucleobase sequence. In certain embodiments, a single-stranded antisense agent comprises an antisense compound, which comprises a modified oligonucleotide and a conjugate group.

In certain embodiments, antisense agents are double-stranded. In certain embodiments, double-stranded antisense agents comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide.

In such embodiments, the thymine nucleobase in the modified oligonucleotide is replaced by a uracil nucleobase. In certain embodiments, a double-stranded antisense agent comprises a conjugate group. In certain embodiments, a double-stranded antisense agent comprises an antisense compound and a sense compound, wherein the sense compound comprises a conjugate group. In certain embodiments, each modified oligonucleotide is 12-30 linked nucleosides in length.

Examples of single-stranded and double-stranded antisense agents include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, an antisense agent described herein comprises an oligonucleotide having a nucleobase sequence that, when written in the 5′ to 3′ direction, comprises the reverse complement of the target segment of a target nucleic acid to which it is targeted.

In certain embodiments, an antisense agent, antisense compound, or sense compound described herein comprises an oligonucleotide consisting of 10 to 30 linked nucleosides. In certain embodiments, the oligonucleotide consists of 12 to 30 linked nucleosides. In certain embodiments, the oligonucleotide consists of 12 to 22 linked nucleosides. In certain embodiments, the oligonucleotide consists of 14 to 30 linked nucleosides. In certain embodiments, the oligonucleotide consists of 14 to 20 linked nucleosides. In certain embodiments, the oligonucleotide consists of 15 to 30 linked nucleosides. In certain embodiments, the oligonucleotide consists of 15 to 20 linked nucleosides. In certain embodiments, the oligonucleotide consists of 16 to 30 linked nucleosides. In certain embodiments, the oligonucleotide consists of 16 to 20 linked nucleosides. In certain embodiments, the oligonucleotide consists of 17 to 30 linked nucleosides. In certain embodiments, the oligonucleotide consists of 17 to 20 linked nucleosides. In certain embodiments, the oligonucleotide consists of 18 to 30 linked nucleosides. In certain embodiments, the oligonucleotide consists of 18 to 21 linked nucleosides. In certain embodiments, the oligonucleotide consists of 18 to 20 linked nucleosides. In certain embodiments, the oligonucleotide consists of 20 to 30 linked nucleosides. In certain embodiments, oligonucleotides consist of 12 to 30 linked nucleosides, 14 to 30 linked nucleosides, 14 to 20 linked nucleosides, 15 to 30 linked nucleosides, 15 to 20 linked nucleosides, 16 to 30 linked nucleosides, 16 to 20 linked nucleosides, 17 to 30 linked nucleosides, 17 to 20 linked nucleosides, 18 to 30 linked nucleosides, 18 to 20 linked nucleosides, 18 to 21 linked nucleosides, 20 to 30 linked nucleosides, or 12 to 22 linked nucleosides. In certain embodiments, an oligonucleotide consists of 14 linked nucleosides. In certain embodiments, an oligonucleotide consists of 16 linked nucleosides. In certain embodiments, an oligonucleotide consists of 17 linked nucleosides. In certain embodiments, an oligonucleotide consists of 18 linked nucleosides. In certain embodiments, an oligonucleotide consists of 19 linked nucleosides. In certain embodiments, an oligonucleotide consists of 20 linked nucleosides. In other embodiments, an oligonucleotide consists of 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides. In certain such embodiments, an oligonucleotide consists of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleosides, or a range defined by any two of the above values. In certain embodiments, the oligonucleotide is a modified oligonucleotide. In certain embodiments, the oligonucleotide is an antisense oligonucleotide. In certain embodiments, the oligonucleotide is a sense oligonucleotide.

In certain embodiments, antisense agents described herein are interfering RNA compounds (RNAi), which include double-stranded RNA compounds (also referred to as short-interfering RNA or siRNA) and single-stranded RNAi compounds (or ssRNAi). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics.

In certain embodiments, a double-stranded antisense agent comprises a first oligonucleotide comprising the nucleobase sequence complementary to a target region of a NOX4 nucleic acid and a second oligonucleotide. In certain embodiments, the double-stranded compound comprises ribonucleotides in which the first oligonucleotide has uracil (U) in place of thymine (T) and is complementary to a target region. In certain embodiments, a double-stranded compound comprises (i) a first oligonucleotide comprising a nucleobase sequence complementary to a target region of a NOX4 nucleic acid, and (ii) a second oligonucleotide. In certain embodiments, the double-stranded antisense agent comprises one or more modified nucleotides in which the 2′ position in the sugar contains a halogen (such as fluorine group; 2′-F) or contains an alkoxy group (such as a methoxy group; 2′-OMe). In certain embodiments, the double-stranded antisense agent comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or contiguous nucleobases along a strand of the dsRNA compound. In certain embodiments, the double-stranded antisense agent comprises one or more linkages between adjacent nucleotides other than a naturally-occurring phosphodiester linkage. Examples of such linkages include phosphoramide, phosphorothioate, and phosphorodithioate linkages. The double-stranded compounds may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000. In certain embodiments, the first oligonucleotide of the double-stranded antisense agent is an siRNA guide strand and the second oligonucleotide of the double-stranded compound is an siRNA passenger strand. In certain embodiments, the second oligonucleotide of the double-stranded antisense agent is complementary to the first oligonucleotide. In certain embodiments, each strand of the double-stranded antisense agent consists of 16, 17, 18, 19, 20, 21, 22, or 23 linked nucleosides.

In certain embodiments, a single-stranded antisense agent described herein can comprise any of the oligonucleotide sequences targeted to NOX4 described herein. In certain embodiments, such a single-stranded antisense agent is a single-stranded RNAi (ssRNAi) agent. In certain embodiments, a ssRNAi agent comprises the nucleobase sequence complementary to a target region of a NOX4 nucleic acid. In certain embodiments, the ssRNAi agent comprises ribonucleotides in which uracil (U) is in place of thymine (T). In certain embodiments, ssRNAi agent comprises a nucleobase sequence complementary to a target region of a NOX4 nucleic acid. In certain embodiments, a ssRNAi agent comprises one or more modified nucleotides in which the 2′ position in the sugar contains a halogen (such as fluorine group; 2′-F) or contains an alkoxy group (such as a methoxy group; 2′-OMe). In certain embodiments, a ssRNAi agent comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the ssRNAi agent. In certain embodiments, the ssRNAi agent comprises one or more linkages between adjacent nucleotides other than a naturally-occurring phosphodiester linkage. Examples of such linkages include phosphoramide, phosphorothioate, and phosphorodithioate linkages. The ssRNAi agents may also be chemically modified nucleic acid molecules as taught in U.S. Pat. No. 6,673,661. In other embodiments, the ssRNAi agent contains a capped strand, as disclosed, for example, by WO 00/63364, filed Apr. 19, 2000. In certain embodiments, the ssRNAi agent consists of 16, 17, 18, 19, 20, 21, 22, or 23 linked nucleosides.

In certain embodiments, antisense agents described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as a or R such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

### Certain Mechanisms

In certain embodiments, antisense agents described herein selectively affect one or more target nucleic acid. Such selective antisense agents comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of an antisense agent described herein to a target nucleic acid results in recruitment of a protein that cleaves the target nucleic acid. For example, certain antisense agents described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, antisense agents described herein are sufficiently “DNA-like” to elicit RNase H activity. Further, in certain embodiments, one or more non-DNA-like nucleoside in the gap of a gapmer is tolerated.

In certain antisense activities, antisense agents described herein or a portion of the antisense agent is loaded into an RNA-induced silencing complex (RISC), ultimately resulting in cleavage of the target nucleic acid. For example, certain antisense agents described herein result in cleavage of the target nucleic acid by Argonaute. In certain embodiments, antisense agents that are loaded into RISC are RNAi agents. RNAi agents may be double-stranded (siRNA) or single-stranded (ssRNAi).

In certain embodiments, hybridization of antisense agents described herein to a target nucleic acid does not result in recruitment of a protein that cleaves that target nucleic acid. In certain such embodiments, hybridization of the antisense agents to the target nucleic acid results in alteration of splicing of the target nucleic acid. In certain embodiments, hybridization of the antisense agents to a target nucleic acid results in inhibition of a binding interaction between the target nucleic acid and a protein or other nucleic acid. In certain such embodiments, hybridization of the antisense agents to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or individual.

### Target Nucleic Acids, Target Regions and Nucleotide Sequences

In certain embodiments, antisense agents described herein comprise or consist of an oligonucleotide comprising a region that is complementary to a NOX4 nucleic acid.

Nucleotide sequences that encode NOX4 include, without limitation, the following RefSeq Nos.: GENBANK Accession No. NT_039433.8, truncated from nucleosides 4994000 to U.S. Pat. No. 5,149,000 (incorporated by reference, disclosed herein as SEQ ID NO: 1); GENBANK Accession No. NM_001285833.1 (incorporated by reference, disclosed herein as SEQ ID NO: 2); GENBANK Accession No. NM_015760.5 (incorporated by reference, disclosed herein as SEQ ID NO: 3), the complement of GENBANK Accession No. NT_033899.9 truncated from 1320000 to U.S. Pat. No. 1,588,000 (incorporated by reference, disclosed herein as SEQ ID NO: 4); and GENBANK Accession No. NM_016931.4 (incorporated by reference, disclosed herein as SEQ ID NO: 5).

### Hybridization

In some embodiments, hybridization occurs between an antisense agent disclosed herein and a NOX4 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense agents provided herein are specifically hybridizable with a NOX4 nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methylcytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, antisense agents described herein comprise or consist of modified oligonucleotides. In certain embodiments, antisense agents described herein are antisense compounds. Non-complementary nucleobases between an oligonucleotide and a NOX4 nucleic acid may be tolerated provided that the oligonucleotide remains able to specifically hybridize to a target nucleic acid. Moreover, an oligonucleotide may hybridize over one or more segments of a NOX4 nucleic acid such that intervening or adjacent segments are not involved in the hybridization event (e.g., a loop structure, mismatch or hairpin structure).

In certain embodiments, an oligonucleotide provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a NOX4 nucleic acid, a target region, target segment, or specified portion thereof. Percent complementarity of an oligonucleotide with a target nucleic acid can be determined using routine methods.

For example, an oligonucleotide in which 18 of 20 nucleobases of the oligonucleotide are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an oligonucleotide which is 18 nucleobases in length having four non-complementary nucleobases which are flanked by two regions of complete complementarity with the target nucleic acid would have 77.8% overall complementarity with the target nucleic acid and would thus fall within the scope of the present invention. Percent complementarity of an oligonucleotide with a region of a target nucleic acid can be determined routinely using BLAST programs (basic local alignment search tools) and PowerBLAST programs known in the art (Altschul et al., J Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, oligonucleotides described herein, or specified portions thereof, are fully complementary (i.e. 100% complementary) to a target nucleic acid, or specified portion thereof. For example, an oligonucleotide may be fully complementary to a NOX4 nucleic acid, or a target region, or a target segment or target sequence thereof. As used herein, “fully complementary” means each nucleobase of an oligonucleotide is capable of precise base pairing with the corresponding nucleobases of a target nucleic acid. For example, a 20 nucleobase oligonucleotide is fully complementary to a target sequence that is 400 nucleobases long, so long as there is a corresponding 20 nucleobase portion of the target nucleic acid that is fully complementary to the oligonucleotide. Fully complementary can also be used in reference to a specified portion of the first and/or the second nucleic acid. For example, a 20 nucleobase portion of a 30 nucleobase oligonucleotide can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the nucleobase portion of the oligonucleotide. At the same time, the entire 30 nucleobase oligonucleotide may or may not be fully complementary to the target sequence, depending on whether the remaining 10 nucleobases of the oligonucleotide are also complementary to the target sequence.

In certain embodiments, antisense agents described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense agent is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the oligonucleotide. Alternatively, the non-complementary nucleobase or nucleobases may be at an internal position of the oligonucleotide. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, oligonucleotides described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a NOX4 nucleic acid, or specified portion thereof.

In certain embodiments, oligonucleotides described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a NOX4 nucleic acid, or specified portion thereof.

In certain embodiments, oligonucleotides described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an oligonucleotide. In certain embodiments, the oligonucleotides are complementary to at least an 8 nucleobase portion of a target segment. In certain embodiments, the oligonucleotides are complementary to at least a 9 nucleobase portion of a target segment. In certain embodiments, the oligonucleotides are complementary to at least a 10 nucleobase portion of a target segment. In certain embodiments, the oligonucleotides are complementary to at least an 11 nucleobase portion of a target segment. In certain embodiments, the oligonucleotides are complementary to at least a 12 nucleobase portion of a target segment. In certain embodiments, the oligonucleotides are complementary to at least a 13 nucleobase portion of a target segment. In certain embodiments, the oligonucleotides are complementary to at least a 14 nucleobase portion of a target segment. In certain embodiments, the oligonucleotides are complementary to at least a 15 nucleobase portion of a target segment. In certain embodiments, the oligonucleotides are complementary to at least a 16 nucleobase portion of a target segment. Also contemplated are oligonucleotides that are complementary to at least a 9, 10, 11, 12, 13, 14, 15, 16, 17, 18, 19, 20, or more nucleobase portion of a target segment, or a range defined by any two of these values.

### Identity

The oligonucleotides provided herein may also have a defined percent identity to a particular nucleotide sequence, SEQ ID NO, or compound represented by a specific ION number, or portion thereof. An oligonucleotide is identical to a sequence disclosed herein if it has the same nucleobase pairing ability. For example, an RNA which contains uracil in place of thymidine in a disclosed DNA sequence would be considered identical to the DNA sequence since both uracil and thymidine pair with adenine. Shortened and lengthened versions of the oligonucleotides described herein as well as oligonucleotides having non-identical bases relative to the oligonucleotides provided herein also are contemplated. The non-identical bases may be adjacent to each other or dispersed throughout the oligonucleotide. Percent identity of an oligonucleotide is calculated according to the number of bases that have identical base pairing relative to the sequence to which it is being compared.

### Certain Modified Oligonucleotides

In certain embodiments, antisense agents and antisense compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

### A. Modified Nucleosides

Modified nucleosides comprise a modified sugar moiety or a modified nucleobase or both a modifed sugar moiety and a modified nucleobase.

### 1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: F, OCH₃ (“OMe” or “O-methyl”), and O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂₀N(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂₀N(CH₃)₂ (“DMAOE”), 2′-O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂ (“DMAOE”), 2′-O(CH₂)₂O(CH₂)₂N(CH₃)₂ (“DMAEOE”), and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
   each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJI, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 91999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

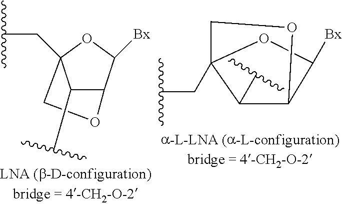

α-L-methyleneoxy (4′-CH₂—O—2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see e.g., Leumann, C J. Bioorg. & Med. Chem. 2002, 10, 841-854), fluoro HNA:

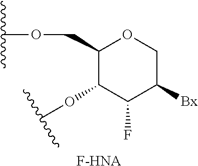

(“F-HNA”, see e.g., Swayze et al., U.S. Pat. No. 8,088,904; Swayze et al., U.S. Pat. No. 8,440,803; Swayze et al., U.S.; and Swayze et al., U.S. Pat. No. 9,005,906, F-HNA can also be referred to as a F-THP or 3′-fluoro tetrahydropyran), and nucleosides comprising additional modified THP compounds having the formula:

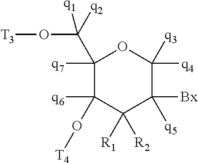

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

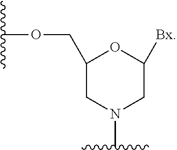

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### 2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to oligonucleotides described herein.

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi-dines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, modified oligonucleotides comprise one or more modified nucleobases. In certain embodiments, the modified nucleobase is 5-methylcytosine. In certain embodiments, each cytosine is a 5-methylcytosine.

### 3. Modified Internucleoside Linkages

The naturally occurring internucleoside linkage of RNA and DNA is a 3′ to 5′ phosphodiester linkage. In certain embodiments, oligonucleotides described herein having one or more modified, i.e. non-naturally occurring, internucleoside linkages are often selected over oligonucleotides having naturally occurring internucleoside linkages because of desirable properties such as, for example, enhanced cellular uptake, enhanced affinity for target nucleic acids, and increased stability in the presence of nucleases.

In certain embodiments, oligonucleotides comprise one or more modified internucleoside linkages. In certain embodiments, the modified internucleoside linkages are phosphorothioate linkages. In certain embodiments, each internucleoside linkage of the oligonucleotide is a phosphorothioate internucleoside linkage.

In certain embodiments, oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O—5′), amide-3 (3′-CH₂—C(═O)—N(H)-5′), amide-4 (3′-CH₂—N(H)—C(═O)-5′), formacetal (3′-O—CH₂—O—5′), methoxypropyl, and thioformacetal (3′-S—CH₂—O—5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, antisense agents and antisense compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

### 2. Certain Nucleobase Motifs

In certain embodiments, antisense agents and antisense compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, antisense agents and antisense compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphodiester internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

### Certain Conjugated Antisense Agents and Antisense Compounds

In certain embodiments, antisense agents and antisense compounds described herein comprise or consist of an oligonucleotide (modified or unmodified) and, optionally, one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides. In certain embodiments, the antisense agent is an RNAi agent comprising a conjugate group. In certain embodiments, the RNAi agent comprises an antisense compound and a sense compound, wherein the sense compound comprises a conjugate group. In certain embodiments, the sense compound comprises a sense oligonucleotide and a conjugate group attached to the sense oligonucleotide. In certain embodiments, the conjugate group is attached to the 3′ end of the sense oligonucleotide.

In certain embodiments, the oligonucleotide is modified. In certain embodiments, the oligonucleotide has a nucleobase sequence that is complementary to a target nucleic acid. In certain embodiments, oligonucleotides are complementary to a messenger RNA (mRNA). In certain embodiments, oligonucleotides are complementary to a pre-mRNA. In certain embodiments, oligonucleotides are complementary to a sense transcript.

Examples of terminal groups include but are not limited to conjugate groups, capping groups, phosphate moieties, protecting groups, modified or unmodified nucleosides, and two or more nucleosides that are independently modified or unmodified.

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain antisense agents and antisense compounds, the conjugate linker is a single chemical bond (i.e., the conjugate moiety is attached directly to an oligonucleotide through a single bond). In certain antisense agents and antisense compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which an antisense agent or antisense compound comprises an oligonucleotide consisting of a specified number or range of linked nucleosides and/or a specified percent complementarity to a reference nucleic acid and the antisense agent or antisense compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an antisense agent or antisense compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an antisense agent or antisense compound is more than 30. Alternatively, an antisense agent or antisense compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances antisense agents or antisense compounds comprising a particular conjugate moiety are better taken up by a particular cell type, but once the antisense agent or antisense compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the antisense agent or antisense compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Certain Cell-Targeting Conjugate Moieties

In certain embodiments, a conjugate group comprises a cell-targeting conjugate moiety. In certain embodiments, a conjugate group has the general formula:

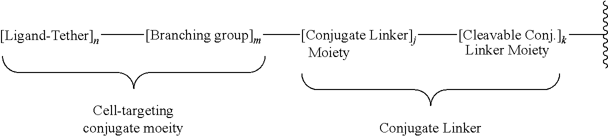

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

Representative United States patents, United States patent application publications, international patent application publications, and other publications that teach the preparation of certain of the above noted conjugate groups, compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011, 19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770.

In certain embodiments, antisense agents comprise a conjugate group found in any of the following references: Lee, Carbohydr Res, 1978, 67, 509-514; Connolly et al., J Biol Chem, 1982, 257, 939-945; Pavia et al., Int JPep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., BioorgMed Chem Lett, 2006, 16(19), 5132-5135; Maierhofer et al., BioorgMed Chem, 2007, 15, 7661-7676; Khorev et al., BioorgMed Chem, 2008, 16, 5216-5231; Lee et al., BioorgMed Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., JAm Chem Soc, 2004, 126, 14013-14022; Lee et al., JOrg Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. patent application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In certain embodiments, antisense agents comprising a conjugate group are single-stranded. In certain embodiments, antisense agents comprising a conjugate group are double-stranded.

### Compositions and Methods for Formulating Pharmaceutical Compositions

Antisense agents described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense agent described herein targeted to a NOX4 nucleic acid can be utilized in pharmaceutical compositions by combining the antisense agent with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising an antisense agent targeted to a NOX4 nucleic acid and a pharmaceutically acceptable diluent. In certain embodiments, the pharmaceutically acceptable diluent is water. In certain embodiments, the antisense agent comprises or consists of a modified oligonucleotide provided herein.

Pharmaceutical compositions comprising antisense agents provided herein encompass any pharmaceutically acceptable salts, esters, or salts of such esters, or any other oligonucleotide which, upon administration to a subject, including a human, is capable of providing (directly or indirectly) the biologically active metabolite or residue thereof. Suitable pharmaceutically acceptable salts include, but are not limited to, sodium and potassium salts.

A prodrug can include the incorporation of additional nucleosides at one or both ends of an antisense agent which are cleaved by endogenous nucleases within the body, to form the active compound.

In certain embodiments, the antisense agents or compositions further comprise a pharmaceutically acceptable carrier or diluent.

### Certain Combinations and Combination Therapies

In certain embodiments, an antisense agent described herein is co-administered with one or more secondary agents. In certain embodiments, such second agents are designed to treat the same disease, disorder, or condition as the first agent described herein. In certain embodiments, such second agents are designed to treat a different disease, disorder, or condition as the first agent described herein. In certain embodiments, a first agent is designed to treat an undesired side effect of a second agent. In certain embodiments, second agents are co-administered with the antisense agent to treat an undesired effect of the antisense agent. In certain embodiments, such second agents are designed to treat an undesired side effect of one or more pharmaceutical compositions as described herein. In certain embodiments, second agents are co-administered with the antisense agent to produce a combinational effect. In certain embodiments, second agents are co-administered with the antisense agent to produce a synergistic effect. In certain embodiments, the co-administration of the antisense and second agents permits use of lower dosages than would be required to achieve a therapeutic or prophylactic effect if the agents were administered as independent therapy.

In certain embodiments, a secondary agent is a vasodilator, such as, for example Treprostinil, prostacyclin, or an endothelin receptor antagonist. In certain embodiments, a secondary agent is a diuretic. In certain embodiments, a secondary agent is a short- or long-acting bronchodilator, a steroid, or a PDE4 inhibitor. In certain embodiments, a secondary agent is oxygen.

### EXAMPLES

### Non-Limiting Disclosure and Incorporation by Reference

While certain antisense agents, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Effect of 3-10-3 cEt, Uniform Phosphorothioate Modified Oligonucleotides on Mouse NOX4 RNA In Vitro, Single Dose

Modified oligonucleotides complementary to mouse NOX4 nucleic acid were designed and tested for their single dose effects on NOX4 RNA in vitro. The modified oligonucleotides were tested in a series of experiments that had the same culture conditions.

The modified oligonucleotides in the table below are 3-10-3 cEt modified oligonucleotides with uniform phosphorothioate internucleoside linkages. The modified oligonucleotides are 16 nucleosides in length, wherein the central gap segment consists of ten 2′-β-D-deoxynucleosides, and wherein the 5′ and 3′ wing segments each consist of three cEt nucleosides. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO: 1 (GENBANK Accession No. NT_039433.8, truncated from nucleosides 4994000 to 5149000), to SEQ ID NO: 2 (GENBANK Accession No. NM_001285833.1), or to both. “N/A” indicates that the modified oligonucleotide is not 100% complementary to that particular target nucleic acid sequence.

Cultured b.END cells were treated with modified oligonucleotide at a concentration of 4000 nM by electroporation at a density of 20,000 cells per well. After a treatment period of approximately 24 hours, total RNA was isolated from the cells and NOX4 RNA levels were measured by quantitative real-time RT-PCR. NOX4 RNA levels were measured by mouse primer-probe set RTS35168 (forward sequence TCCTACTGAAACCAAAGCAACA, designated herein as SEQ ID NO: 6; reverse sequence AATGAAGGGCAGAATCTCAGAG, designated herein as SEQ ID NO: 7; probe sequence AGACTGGACAGAACGATTCCGGGA, designated herein as SEQ ID NO: 8). NOX4 RNA levels were normalized to total RNA content, as measured by RIBOGREEN®. Reduction of NOX4 RNA is presented in the table below as percent NOX4 RNA relative to the amount ofNOX4 RNA in untreated control cells (% UTC). The values marked with a “t” indicate that the modified oligonucleotide is complementary to the amplicon region of the primer-probe set. Additional assays may be used to measure the potency and efficacy of the modified oligonucleotides complementary to the amplicon region.

Each separate experimental analysis described in this example is identified by a letter ID in the table column below labeled “AID” (Analysis ID).

TABLE 1
Reduction of mouse NOX4 RNA by 3-10-3 cEt modified oligonucleotides with uniform
phosphorothioate internucleoside linkages at a concentration of 4000 nM in b.END cells

| | SEQ ID | SEQ ID | SEQ ID | SEQ ID | | | | |
|---|---|---|---|---|---|---|---|---|
| | No: 1 | No: 1 | No: 2 | No: 2 | | NOX4 | | |
| Compound | Start | Stop | Start | Stop | | (% | | SEQ |
| Number | Site | Site | Site | Site | Sequence (5′ to 3′) | UTC) | AID | ID NO |
| 805993 | 1341 | 1356 | 15 | 30 | TCCCACCGCGCTGCTC | 70 | A | 12 |
| 805997 | 1441 | 1456 | 115 | 130 | GGCAGCAGATCCAGGG | 67 | A | 13 |
| 806001 | 1525 | 1540 | 199 | 214 | CTCCAGGACACCGCCA | 37 | A | 14 |
| 806005 | 2186 | 2201 | 275 | 290 | CCAGAAAAGTAGAACG | 14 | A | 15 |
| 806013 | 50510 | 50525 | 395 | 410 | AAAGGATGAGGCTGCA | 13 | A | 16 |
| 806017 | N/A | N/A | 452 | 467 | TGCTAGGGACCTTCTG | 31 | A | 17 |
| 806021 | 52190 | 52205 | 515 | 530 | AGATAGTTACACCACA | 41 | A | 18 |
| 806029 | 59591 | 59606 | 618 | 633 | TGGTATCTTGCTGCAT | 29 | A | 19 |
| 806033 | 68758 | 68773 | 685 | 700 | CACCACCATGCAGACA | 26 | A | 20 |
| 806037 | 71911 | 71926 | 744 | 759 | ATATCATAATTAGAAA | 97 | A | 21 |
| 806041 | 71968 | 71983 | 801 | 816 | ACATGCAACAGCAGCA | 24 | A | 22 |
| 806045 | 76252 | 76267 | 851 | 866 | CAGGAGGGTGAGTGTC | 58 | A | 23 |
| 806049 | 76332 | 76347 | 931 | 946 | CAAAGATCCATGAAAA | 53 | A | 24 |
| 806053 | 76391 | 76406 | 990 | 1005 | AGGCAAATCTTCACAA | 22 | A | 25 |
| 806057 | 78317 | 78332 | 1046 | 1061 | GTCCAGAAATCCAAAT | 37 | A | 26 |
| 806061 | 78377 | 78392 | 1106 | 1121 | TGACAGGTTTGTTGCT | 25 | A | 27 |
| 806065 | 78426 | 78441 | 1155 | 1170 | ATACGGAGTTCCATTA | 31 | A | 28 |
| 806069 | 78615 | 78630 | 1209 | 1224 | GGGCAATGGAGAATAA | 26 | A | 29 |
| 806073 | 93640 | 93655 | 1390 | 1405 | TGCTTTGGTTTCAGTA | 7+ | A | 30 |
| 806077 | 115420 | 115435 | 1460 | 1475 | ATGGAGGCAGTAGCAA | 11+ | A | 31 |
| 806081 | 115474 | 115489 | 1514 | 1529 | ACTTAGGGTAATTTCT | 55 | A | 32 |
| 806085 | 124734 | 124749 | 1566 | 1581 | TCATAGTTCAGTGACT | 34 | A | 33 |
| 806089 | 124791 | 124806 | 1623 | 1638 | TTTAGTATCGATGCAA | 40 | A | 34 |
| 806093 | 126629 | 126644 | 1680 | 1695 | ACCCAGATAAAGTACA | 44 | A | 35 |
| 806097 | 126683 | 126698 | 1734 | 1749 | TGCAACACACAGAGTA | 26 | A | 36 |
| 806105 | 130881 | 130896 | 1847 | 1862 | TCGAATTCAGTGTGTG | 14 | A | 37 |
| 806109 | 130937 | 130952 | 1903 | 1918 | ACATTTTGCTATTTCA | 33 | A | 38 |
| 806113 | 151488 | 151503 | 1966 | 1981 | ATGAAGAGTCTTGGAA | 30 | A | 39 |
| 806117 | 151538 | 151553 | 2016 | 2031 | TTATTGTATTCAAATT | 106 | A | 40 |
| 806121 | 151586 | 151601 | 2064 | 2079 | CTCTTTAAAGTCCCCA | 17 | A | 41 |
| 806125 | 151639 | 151654 | 2117 | 2132 | GCACAGCTGTTTGATT | 61 | A | 42 |
| 806129 | 151714 | 151729 | 2192 | 2207 | TGCCATTTTGCCACAT | 15 | A | 43 |
| 806133 | 151776 | 151791 | 2254 | 2269 | ATCAAAATGTGAATCA | 29 | A | 44 |
| 806137 | 151834 | 151849 | 2312 | 2327 | CTTGAATTTCCCATAA | 26 | A | 45 |
| 806141 | 151893 | 151908 | 2371 | 2386 | AAAGAGGGCTGCAGCT | 25 | A | 46 |
| 806149 | 152001 | 152016 | 2479 | 2494 | ATCCAAATGAAAGCTA | 44 | A | 47 |
| 806153 | 152062 | 152077 | 2540 | 2555 | ACCCGTATCGATACAA | 20 | A | 48 |
| 806157 | 152108 | 152123 | 2586 | 2601 | CTGCAGCGAGGCAGGA | 47 | A | 49 |
| 806161 | 152160 | 152175 | 2638 | 2653 | AGATACTACATCCACA | 17 | A | 50 |
| 806165 | 152219 | 152234 | 2697 | 2712 | CAAGATTGGCTAAGGG | 23 | A | 51 |
| 806169 | 152268 | 152283 | 2746 | 2761 | CTAAATTGAACAAATC | 91 | A | 52 |
| 806173 | 152317 | 152332 | 2795 | 2810 | GGTTTTTAAGGATAAG | 18 | A | 53 |
| 806177 | 152381 | 152396 | 2859 | 2874 | TCAAAGTGAGTCAAAG | 32 | A | 54 |
| 806181 | 152435 | 152450 | 2913 | 2928 | TGTAGAAAGCAAAGCA | 10 | A | 55 |
| 806185 | 152487 | 152502 | 2965 | 2980 | TATTAGTGATTCTAAA | 98 | A | 56 |
| 806189 | 152534 | 152549 | 3012 | 3027 | GACTTAAATCTAGTAA | 58 | A | 57 |
| 806193 | 152584 | 152599 | 3062 | 3077 | CCTGAATATTAAAAAA | 76 | A | 58 |
| 806197 | 152636 | 152651 | 3114 | 3129 | ATAAACTGTATGAATA | 69 | A | 59 |
| 806201 | 152687 | 152702 | 3165 | 3180 | AGCAATGTGTCCTATT | 10 | A | 60 |
| 806205 | 152731 | 152746 | 3209 | 3224 | TGATATACAAAAGATT | 112 | A | 61 |
| 806209 | 152779 | 152794 | 3257 | 3272 | ACTGAATCACATGAAA | 61 | A | 62 |
| 806213 | 152832 | 152847 | 3310 | 3325 | AATATACAGGTGGTTA | 24 | A | 63 |
| 806217 | 152885 | 152900 | 3363 | 3378 | CATAGGAGGGATAGGA | 47 | A | 64 |
| 806221 | 152931 | 152946 | 3409 | 3424 | AGTTTATGTCTCCACA | 8 | A | 65 |
| 806225 | 152989 | 153004 | 3467 | 3482 | TGTAGTATGTGTTTTG | 11 | A | 66 |
| 806229 | 153060 | 153075 | 3538 | 3553 | AATCAGTATTGCACAT | 19 | A | 67 |
| 806233 | 153110 | 153125 | 3588 | 3603 | GAAATGTGATGAATTC | 41 | A | 68 |
| 806237 | 153159 | 153174 | 3637 | 3652 | ACACAAGTCTGAAATA | 35 | A | 69 |
| 806241 | 153203 | 153218 | 3681 | 3696 | TGCACTGGAGTTTAGA | 26 | A | 70 |
| 806245 | 153255 | 153270 | 3733 | 3748 | AATTTATTCCTGTTCA | 25 | A | 71 |
| 806249 | 153306 | 153321 | 3784 | 3799 | ATAGAATCTAAAAAGA | 98 | A | 72 |
| 806253 | 153357 | 153372 | 3835 | 3850 | TATTACCATCAAAATT | 91 | A | 73 |
| 806257 | N/A | N/A | 342 | 357 | CACAATCGCCCAACAT | 67 | A | 74 |
| 806261 | 81847 | 81862 | 1306 | 1321 | GAAACTGTATATCCAT | 10 | A | 75 |
| 806265 | 86397 | 86412 | 1361 | 1376 | AGGCATGGTTCATCTT | 35 | A | 76 |
| 806269 | 18518 | 18533 | N/A | N/A | TATTGGTAAAAACTGG | 34 | A | 77 |
| 806273 | 32930 | 32945 | N/A | N/A | ACTTACATGGACACAT | 32 | A | 78 |
| 806277 | 44893 | 44908 | N/A | N/A | AGTATTGGAATTCAGG | 7 | A | 79 |
| 806281 | 48551 | 48566 | N/A | N/A | GTCTGACCCTTACCTT | 26 | A | 80 |
| | 68812 | 68827 | | | | | | |
| 806285 | 72496 | 72511 | N/A | N/A | GTTCATACTGGGTTAC | 5 | A | 81 |
| 806289 | 93921 | 93936 | N/A | N/A | ACTGACAATAAGAGGC | 8 | A | 82 |
| 806293 | 96139 | 96154 | N/A | N/A | GCCTTTAAATACAAAA | 40 | A | 83 |
| 806297 | 124106 | 124121 | N/A | N/A | CTTAACTACACAGAAA | 67 | A | 84 |
| 806301 | 141137 | 141152 | N/A | N/A | CCCCACCGAGGGATCT | 87 | A | 85 |
| 805994 | 1352 | 1367 | 26 | 41 | CTCCACCCGGCTCCCA | 61 | B | 86 |
| 805998 | 1457 | 1472 | 131 | 146 | AGCGATCAGTGGCCGG | 43 | B | 87 |
| 806006 | 2197 | 2212 | 286 | 301 | AGGAAGGTTTTCCAGA | 28 | B | 88 |
| 806010 | 50476 | 50491 | 361 | 376 | TGCAGATGCTCTGCTT | 50 | B | 89 |
| 806014 | 50521 | 50536 | 406 | 421 | GCACATAGGTAAAAGG | 21 | B | 90 |
| 806018 | 52147 | 52162 | 472 | 487 | CAACAATCTTCTTGTT | 63 | B | 91 |
| 806026 | 59550 | 59565 | 577 | 592 | GTTCACTGAAAAGTTG | 23 | B | 92 |
| 806030 | 61405 | 61420 | 639 | 654 | AGCTTTCTGGGATCCT | 24 | B | 93 |
| 806034 | 68769 | 68784 | 696 | 711 | AGGAACAATACCACCA | 7 | B | 94 |
| 806042 | N/A | N/A | 812 | 827 | AACCACCTGAAACATG | 52 | B | 95 |
| 806046 | 76263 | 76278 | 862 | 877 | ACTAATGCAGCCAGGA | 18 | B | 96 |
| 806050 | 76346 | 76361 | 945 | 960 | GAAAACCCTCGAGGCA | 19 | B | 97 |
| 806054 | 76407 | 76422 | 1006 | 1021 | GAACTTGGGTTCTTCC | 53 | B | 98 |
| 806058 | 78331 | 78346 | 1060 | 1075 | ATAAAGGCACAAAGGT | 23 | B | 99 |
| 806062 | 78390 | 78405 | 1119 | 1134 | ACTGAGATGATGGTGA | 16 | B | 100 |
| 806066 | 78447 | 78462 | 1176 | 1191 | GCTTTAAAGTTTTCTT | 46 | B | 101 |
| 806070 | 78633 | 78648 | 1227 | 1242 | TCTAATGCTGATACAC | 13 | B | 102 |
| 806074 | 93653 | 93668 | 1403 | 1418 | GGACACCAAATGTTGC | 2+ | B | 103 |
| 806078 | 115438 | 115453 | 1478 | 1493 | TCTCAGAGTCTTGACT | 33+ | B | 104 |
| 806082 | N/A | N/A | 1525 | 1540 | ATCAATGTATAACTTA | 39 | B | 105 |
| 806086 | 124745 | 124760 | 1577 | 1592 | AGAGACTAACTTCATA | 26 | B | 106 |
| 806090 | N/A | N/A | 1636 | 1651 | ATCCAGTAGAGTGTTT | 68 | B | 107 |
| 806094 | 126650 | 126665 | 1701 | 1716 | AATGATTGGATGTCTC | 38 | B | 108 |
| 806106 | 130892 | 130907 | 1858 | 1873 | AATGAAAAGTCTCGAA | 54 | B | 109 |
| 806110 | N/A | N/A | 1924 | 1939 | TCCAACTGTTTTCCCT | 24 | B | 110 |
| 806114 | 151501 | 151516 | 1979 | 1994 | GGTTACTCAAACTATG | 11 | B | 111 |
| 806118 | 151550 | 151565 | 2028 | 2043 | CTGAAGGATTCTTTAT | 50 | B | 112 |
| 806122 | 151598 | 151613 | 2076 | 2091 | TTGCACTTGTTCCTCT | 16 | B | 113 |
| 806126 | 151666 | 151681 | 2144 | 2159 | AGCAAACCCTTGGTAT | 15 | B | 114 |
| 806130 | 151725 | 151740 | 2203 | 2218 | CAGATTAACCATGCCA | 6 | B | 115 |
| 806134 | 151787 | 151802 | 2265 | 2280 | TTAATTTGCTCATCAA | 13 | B | 116 |
| 806138 | 151846 | 151861 | 2324 | 2339 | TGTAAGAGACTTCTTG | 32 | B | 117 |
| 806142 | 151905 | 151920 | 2383 | 2398 | CGAAAACAATTTAAAG | 95 | B | 118 |
| 806146 | 151965 | 151980 | 2443 | 2458 | GAAAATGGAGTCTGAG | 31 | B | 119 |
| 806150 | 152023 | 152038 | 2501 | 2516 | TCCCAGTGTTTGCCTG | 33 | B | 120 |
| 806154 | 152073 | 152088 | 2551 | 2566 | AGATATGAAGTACCCG | 12 | B | 121 |
| 806158 | 152119 | 152134 | 2597 | 2612 | GATTAGGAACACTGCA | 25 | B | 122 |
| 806162 | 152171 | 152186 | 2649 | 2664 | CAAACATAAGTAGATA | 47 | B | 123 |
| 806166 | 152230 | 152245 | 2708 | 2723 | ATAATTGTGTCCAAGA | 15 | B | 124 |
| 806170 | 152283 | 152298 | 2761 | 2776 | TACTAAGCCATGGGAC | 27 | B | 125 |
| 806174 | 152330 | 152345 | 2808 | 2823 | TACAAAGCTCTTTGGT | 21 | B | 126 |
| 806178 | 152392 | 152407 | 2870 | 2885 | CTTATCTGAAATCAAA | 62 | B | 127 |
| 806182 | 152451 | 152466 | 2929 | 2944 | GAAGAAGTTCTACTTT | 30 | B | 128 |
| 806186 | 152499 | 152514 | 2977 | 2992 | GTGCAAAAATAATATT | 98 | B | 129 |
| 806190 | 152550 | 152565 | 3028 | 3043 | GATCACATGAACAGGG | 16 | B | 130 |
| 806194 | 152603 | 152618 | 3081 | 3096 | GTAAATTTTCCTTCTG | 13 | B | 131 |
| 806198 | 152647 | 152662 | 3125 | 3140 | AAATTAAGTTGATAAA | 91 | B | 132 |
| 806202 | 152698 | 152713 | 3176 | 3191 | GTACTAATTTCAGCAA | 11 | B | 133 |
| 806206 | 152742 | 152757 | 3220 | 3235 | TTGTAATCATGTGATA | 81 | B | 134 |
| 806210 | 152799 | 152814 | 3277 | 3292 | TAGTATGCTTGTCTGT | 12 | B | 135 |
| 806214 | 152847 | 152862 | 3325 | 3340 | ATATATGTGTAACTAA | 76 | B | 136 |
| 806218 | 152897 | 152912 | 3375 | 3390 | TTGCACATTGCCCATA | 20 | B | 137 |
| 806222 | 152942 | 152957 | 3420 | 3435 | TATGATGTCATAGTTT | 29 | B | 138 |
| 806226 | 153016 | 153031 | 3494 | 3509 | TATGAGGTTTATTTTT | 69 | B | 139 |
| 806230 | 153071 | 153086 | 3549 | 3564 | GATTGGTAAGGAATCA | 38 | B | 140 |
| 806234 | 153121 | 153136 | 3599 | 3614 | CCTGAATTTTTGAAAT | 77 | B | 141 |
| 806238 | 153170 | 153185 | 3648 | 3663 | CTCAACATAGCACACA | 27 | B | 142 |
| 806242 | 153218 | 153233 | 3696 | 3711 | ATAGATCCTTGAACAT | 27 | B | 143 |
| 806246 | 153266 | 153281 | 3744 | 3759 | GATAAAGCTTTAATTT | 69 | B | 144 |
| 806250 | 153320 | 153335 | 3798 | 3813 | CAGTAACCACCACCAT | 13 | B | 145 |
| 806254 | 153360 | 153375 | 3838 | 3853 | ATTTATTACCATCAAA | 57 | B | 146 |
| 806258 | N/A | N/A | 1254 | 1269 | AGCATTGTGAGAGTAA | 31 | B | 147 |
| 806262 | 81858 | 81873 | 1317 | 1332 | CTACAGACAAAGAAAC | 60 | B | 148 |
| 806266 | N/A | N/A | 1372 | 1387 | ACACAGTAAGAAGGCA | 98+ | B | 149 |
| 806270 | 21665 | 21680 | N/A | N/A | TCAGAAATACAGGTGA | 86 | B | 150 |
| 806274 | 33911 | 33926 | N/A | N/A | GATAAAGGACTTACAA | 39 | B | 151 |
| 806278 | 48071 | 48086 | N/A | N/A | GTTACTATCACTCATA | 24 | B | 152 |
| 806282 | 70181 | 70196 | N/A | N/A | ACCATATAAACATTTT | 25 | B | 153 |
| 806286 | 82671 | 82686 | N/A | N/A | GTTTATGAATTCTGAT | 85 | B | 154 |
| 806290 | 93995 | 94010 | N/A | N/A | CTCAATAACACCATGA | 22 | B | 155 |
| 806294 | 102047 | 102062 | N/A | N/A | TCACATTGGCAATTAA | 13 | B | 156 |
| 806298 | 128170 | 128185 | N/A | N/A | AGAACCATCTTTGTTC | 24 | B | 157 |
| | 140404 | 140419 | | | | | | |
| 806302 | 141743 | 141758 | N/A | N/A | AAAGATGTCATGCAAT | 66 | B | 158 |
| 805995 | 1383 | 1398 | 57 | 72 | CTGTACTGAGTCTGGA | 118 | C | 159 |
| 805999 | 1470 | 1485 | 144 | 159 | CAGGACCGAAAGGAGC | 32 | C | 160 |
| 806003 | N/A | N/A | 247 | 262 | CAAATGAGCAGGCAGA | 37 | C | 161 |
| 806007 | 2227 | 2242 | 316 | 331 | TAGTAGTATTCTGGCC | 48 | C | 162 |
| 806011 | 50488 | 50503 | 373 | 388 | GTTCAGGACAGATGCA | 15 | C | 163 |
| 806015 | 50536 | 50551 | 421 | 436 | AGCCAGGACTGTCCGG | 35 | C | 164 |
| 806019 | 52168 | 52183 | 493 | 508 | GTGTAGAGTCTTGCTT | 18 | C | 165 |
| 806023 | N/A | N/A | 539 | 554 | CTACATGCACACCTGA | 18 | C | 166 |
| 806027 | 59567 | 59582 | 594 | 609 | AGGAAATCTTCACTGT | 24 | C | 167 |
| 806031 | N/A | N/A | 658 | 673 | AGGAATGGTTGTGAAG | 42 | C | 168 |
| 806035 | 68780 | 68795 | 707 | 722 | CTGTAACCATGAGGAA | 44 | C | 169 |
| 806039 | 71936 | 71951 | 769 | 784 | GAAGAGGTTGTGAGTA | 30 | C | 170 |
| 806043 | 76226 | 76241 | 825 | 840 | TGATACTTCAACAAAC | 40 | C | 171 |
| 806047 | 76289 | 76304 | 888 | 903 | ATATTCTGGGATGATG | 25 | C | 172 |
| 806051 | 76363 | 76378 | 962 | 977 | AACGATCTTCTAATTT | 42 | C | 173 |
| 806055 | 76418 | 76433 | 1017 | 1032 | AAATGAGCTTGGAACT | 69 | C | 174 |
| 806059 | 78349 | 78364 | 1078 | 1093 | GTAAAGTCTCTCCGCA | 7 | C | 175 |
| 806063 | 78401 | 78416 | 1130 | 1145 | GATGATTGATGACTGA | 10 | C | 176 |
| 806067 | 78458 | 78473 | 1187 | 1202 | GGCCAGGTCTTGCTTT | 58 | C | 177 |
| 806071 | 78647 | 78662 | 1241 | 1256 | TAAATGGGTGGTTTTC | 69 | C | 178 |
| 806075 | 93664 | 93679 | 1414 | 1429 | TACTTTAAAGTGGACA | 27+ | C | 179 |
| 806079 | 115451 | 115466 | 1491 | 1506 | ATGAAGGGCAGAATCT | 22+ | C | 180 |
| 806083 | 124704 | 124719 | 1536 | 1551 | CCAAATGGACCATCAA | 24 | C | 181 |
| 806095 | 126661 | 126676 | 1712 | 1727 | CAAACCACTGGAATGA | 34 | C | 182 |
| 806099 | 129045 | 129060 | 1770 | 1785 | ACAAAGTCAGGTCTGT | 27 | C | 183 |
| 806103 | N/A | N/A | 1819 | 1834 | AATTATCTTCTGAATC | 91 | C | 184 |
| 806111 | 151457 | 151472 | 1935 | 1950 | CAGCAGAAAACTCCAA | 59 | C | 185 |
| 806115 | 151514 | 151529 | 1992 | 2007 | TATGAGTTGTTCCGGT | 15 | C | 186 |
| 806119 | 151563 | 151578 | 2041 | 2056 | CCTAAGGTTTCAGCTG | 26 | C | 187 |
| 806123 | 151614 | 151629 | 2092 | 2107 | TTCTAAGTCTTAGAAA | 124 | C | 188 |
| 806127 | 151677 | 151692 | 2155 | 2170 | TAATCATAAATAGCAA | 89 | C | 189 |
| 806131 | 151753 | 151768 | 2231 | 2246 | AGATATTCTTCAGGGC | 9 | C | 190 |
| 806135 | 151801 | 151816 | 2279 | 2294 | CTAACGGCATTCTTTT | 25 | C | 191 |
| 806139 | 151866 | 151881 | 2344 | 2359 | TGTAAGTGAGTTCAGC | 15 | C | 192 |
| 806143 | 151920 | 151935 | 2398 | 2413 | TTGAATGTGTTCAACC | 8 | C | 193 |
| 806147 | 151976 | 151991 | 2454 | 2469 | TGCACAACCTAGAAAA | 29 | C | 194 |
| 806151 | 152037 | 152052 | 2515 | 2530 | CTACAGTTTCAACCTC | 19 | C | 195 |
| 806155 | 152084 | 152099 | 2562 | 2577 | AGTGAAGCAGCAGATA | 17 | C | 196 |
| 806159 | 152136 | 152151 | 2614 | 2629 | AGCTTTGTGAGCATAT | 13 | C | 197 |
| 806163 | 152182 | 152197 | 2660 | 2675 | AGGATAAGATGCAAAC | 10 | C | 198 |
| 806167 | 152245 | 152260 | 2723 | 2738 | AAACAGTTCTATTAAA | 82 | C | 199 |
| 806171 | 152295 | 152310 | 2773 | 2788 | GCACATTTTCATTACT | 9 | C | 200 |
| 806175 | 152345 | 152360 | 2823 | 2838 | GTGTTTGGTGCAAGAT | 13 | C | 201 |
| 806179 | 152407 | 152422 | 2885 | 2900 | ATGAATTCAGCATCTC | 8 | C | 202 |
| 806183 | 152462 | 152477 | 2940 | 2955 | GAATAAATACTGAAGA | 60 | C | 203 |
| 806187 | 152511 | 152526 | 2989 | 3004 | TGATATCAAACAGTGC | 6 | C | 204 |
| 806191 | 152562 | 152577 | 3040 | 3055 | ATAAACTGATTGGATC | 22 | C | 205 |
| 806195 | 152614 | 152629 | 3092 | 3107 | TTCTTAAGGTAGTAAA | 51 | C | 206 |
| 806199 | 152659 | 152674 | 3137 | 3152 | TAATTATATACCAAAT | 112 | C | 207 |
| 806203 | 152709 | 152724 | 3187 | 3202 | CATAAGTTAAGGTACT | 71 | C | 208 |
| 806207 | 152754 | 152769 | 3232 | 3247 | TAACAGGATTTATTGT | 101 | C | 209 |
| 806211 | 152810 | 152825 | 3288 | 3303 | GCTAAATGTTTTAGTA | 51 | C | 210 |
| 806219 | 152908 | 152923 | 3386 | 3401 | CTACATGAGCATTGCA | 25 | C | 211 |
| 806223 | 152953 | 152968 | 3431 | 3446 | GACAGCATTGCTATGA | 13 | C | 212 |
| 806227 | 153036 | 153051 | 3514 | 3529 | GACAAGCCCAAGTTTT | 35 | C | 213 |
| 806231 | 153082 | 153097 | 3560 | 3575 | AAATAAGTTATGATTG | 99 | C | 214 |
| 806235 | 153132 | 153147 | 3610 | 3625 | TGTAAGTTTGACCTGA | 17 | C | 215 |
| 806239 | 153181 | 153196 | 3659 | 3674 | AAGGAAGTTCACTCAA | 17 | C | 216 |
| 806243 | 153229 | 153244 | 3707 | 3722 | ATGTTTAACATATAGA | 84 | C | 217 |
| 806247 | 153277 | 153292 | 3755 | 3770 | TCTATATGTGTGATAA | 51 | C | 218 |
| 806251 | 153331 | 153346 | 3809 | 3824 | GATTTATACAACAGTA | 33 | C | 219 |
| 806255 | 153363 | 153378 | 3841 | 3856 | AATATTTATTACCATC | 87 | C | 220 |
| 806259 | 81822 | 81837 | 1281 | 1296 | TGCCAGGGCTGCCATC | 13 | C | 221 |
| 806263 | N/A | N/A | 1332 | 1347 | TGGCATGCAGCATCAC | 39 | C | 222 |
| 806267 | 10264 | 10279 | N/A | N/A | GAAACATTGTGTGTGA | 7 | C | 223 |
| 806271 | 27151 | 27166 | N/A | N/A | TGACAAGCCAAATTGA | 26 | C | 224 |
| 806275 | 34580 | 34595 | N/A | N/A | TGGAAGAAGTTAAGAA | 61 | C | 225 |
| 806279 | 53290 | 53305 | N/A | N/A | TAATGATACTCAGCAA | 39 | C | 226 |
| 806283 | 71014 | 71029 | N/A | N/A | GTGCAGATAATGGTGA | 16 | C | 227 |
| 806287 | 86295 | 86310 | N/A | N/A | AGCCAGAAGGGACGAG | 25 | C | 228 |
| 806291 | 95515 | 95530 | N/A | N/A | GACCATTAGGCCTTCC | 12 | C | 229 |
| 806295 | 114599 | 114614 | N/A | N/A | TAGTAAAAGAAGCAAC | 103 | C | 230 |
| 806299 | 128172 | 128187 | N/A | N/A | ATAGAACCATCTTTGT | 15 | C | 231 |
| | 140406 | 140421 | | | | | | |
| 806303 | 148258 | 148273 | N/A | N/A | GTGTACAGGTGAGTGT | 83 | C | 232 |
| 806215 | 152863 | 152878 | 3341 | 3356 | TCCAAGGATAAAAAAA | 61 | C | 233 |
| 805996 | 1398 | 1413 | 72 | 87 | GGCGAGGGTCAAAGGC | 10 | D | 234 |
| 806000 | 1514 | 1529 | 188 | 203 | CGCCATGCCGCCGGCG | 80 | D | 235 |
| 806004 | 2169 | 2184 | 258 | 273 | TTAGGGACAGCCAAAT | 10 | D | 236 |
| 806008 | 2242 | 2257 | 331 | 346 | AACATTTGGTGAATGT | 89 | D | 237 |
| 806012 | 50499 | 50514 | 384 | 399 | CTGCAGTTGAGGTTCA | 17 | D | 238 |
| 806016 | 50547 | 50562 | 432 | 447 | CCTCGAAGATAAGCCA | 70 | D | 239 |
| 806020 | 52179 | 52194 | 504 | 519 | CCACATGTGATGTGTA | 31 | D | 240 |
| 806024 | 59525 | 59540 | 552 | 567 | ACCAAGTGGGCGGCTA | 17 | D | 241 |
| 806028 | 59580 | 59595 | 607 | 622 | TGCATTCAGTTCAAGG | 67 | D | 242 |
| 806032 | 68747 | 68762 | 674 | 689 | AGACACCCGTCAGACC | 11 | D | 243 |
| 806036 | N/A | N/A | 733 | 748 | AGAAACTCTTATTGCG | 8 | D | 244 |
| 806044 | 76237 | 76252 | 836 | 851 | CTACATTTGTCTGATA | 9 | D | 245 |
| 806048 | 76321 | 76336 | 920 | 935 | GAAAATGTTCTGAGAC | 15 | D | 246 |
| 806052 | 76380 | 76395 | 979 | 994 | CACAAGTGTTTTCTGG | 9 | D | 247 |
| 806056 | N/A | N/A | 1034 | 1049 | AAATCCAGGTCTGTGG | 35 | D | 248 |
| 806060 | 78362 | 78377 | 1091 | 1106 | TCCTGATGCATCGGTA | 11 | D | 249 |
| 806064 | 78413 | 78428 | 1142 | 1157 | TTACATCAGAGGGATG | 10 | D | 250 |
| 806068 | N/A | N/A | 1198 | 1213 | AATAATATACTGGCCA | 46 | D | 251 |
| 806076 | 115408 | 115423 | 1448 | 1463 | GCAAATCCCGGAATCG | 0+ | D | 252 |
| 806080 | 115462 | 115477 | 1502 | 1517 | TTCTAGAGTGAATGAA | 70+ | D | 253 |
| 806084 | 124716 | 124731 | 1548 | 1563 | TCAAATGGGCTTCCAA | 27 | D | 254 |
| 806088 | 124779 | 124794 | 1611 | 1626 | GCAAATGGAGTGACTC | 21 | D | 255 |
| 806092 | 126618 | 126633 | 1669 | 1684 | GTACAGTCTTCTTAAC | 39 | D | 256 |
| 806100 | 129057 | 129072 | 1782 | 1797 | AGCTGGATGTTCACAA | 17 | D | 257 |
| 806108 | 130926 | 130941 | 1892 | 1907 | TTTCATCAAATAAAAG | 77 | D | 258 |
| 806112 | 151471 | 151486 | 1949 | 1964 | TAGAACTGGGTCCACA | 22 | D | 259 |
| 806116 | 151527 | 151542 | 2005 | 2020 | AAATTTTGTCCCATAT | 52 | D | 260 |
| 806120 | 151575 | 151590 | 2053 | 2068 | CCCCAGTAGTCTCCTA | 9 | D | 261 |
| 806124 | 151626 | 151641 | 2104 | 2119 | ATTCAGCTGAGTTTCT | 73 | D | 262 |
| 806128 | 151688 | 151703 | 2166 | 2181 | CTCATTTTAAATAATC | 43 | D | 263 |
| 806132 | 151765 | 151780 | 2243 | 2258 | AATCACCACATCAGAT | 38 | D | 264 |
| 806136 | 151822 | 151837 | 2300 | 2315 | ATAAAAATCAACAGCG | 25 | D | 265 |
| 806140 | 151878 | 151893 | 2356 | 2371 | TATCAGCTTCAGTGTA | 22 | D | 266 |
| 806148 | 151990 | 152005 | 2468 | 2483 | AGCTACTCCACTTATG | 29 | D | 267 |
| 806152 | 152050 | 152065 | 2528 | 2543 | ACAAAGTTTTGTGCTA | 11 | D | 268 |
| 806156 | 152097 | 152112 | 2575 | 2590 | CAGGAGAGTCAGTAGT | 33 | D | 269 |
| 806160 | 152149 | 152164 | 2627 | 2642 | CCACATGGGTATAAGC | 12 | D | 270 |
| 806164 | 152193 | 152208 | 2671 | 2686 | TTCCTAGACGGAGGAT | 62 | D | 271 |
| 806168 | 152257 | 152272 | 2735 | 2750 | AAATCGGTGATCAAAC | 39 | D | 272 |
| 806172 | 152306 | 152321 | 2784 | 2799 | ATAAGCTTAAAGCACA | 19 | D | 273 |
| 806176 | 152366 | 152381 | 2844 | 2859 | GGAGAGAGGTCTTGTG | 24 | D | 274 |
| 806180 | 152424 | 152439 | 2902 | 2917 | AAGCAGGGTATCACTC | 21 | D | 275 |
| 806184 | 152473 | 152488 | 2951 | 2966 | AACTGAAATTTGAATA | 133 | D | 276 |
| 806188 | 152523 | 152538 | 3001 | 3016 | AGTAACTCACCGTGAT | 18 | D | 277 |
| 806192 | 152573 | 152588 | 3051 | 3066 | AAAAATAGGTGATAAA | 113 | D | 278 |
| 806196 | 152625 | 152640 | 3103 | 3118 | GAATATCTTCTTTCTT | 12 | D | 279 |
| 806200 | 152670 | 152685 | 3148 | 3163 | GATAGCATTAATAATT | 37 | D | 280 |
| 806204 | 152720 | 152735 | 3198 | 3213 | AGATTACCAAGCATAA | 18 | D | 281 |
| 806208 | 152768 | 152783 | 3246 | 3261 | TGAAATCCAAATAATA | 110 | D | 282 |
| 806212 | 152821 | 152836 | 3299 | 3314 | GGTTATTTGATGCTAA | 4 | D | 283 |
| 806216 | 152874 | 152889 | 3352 | 3367 | TAGGAAAGTGTTCCAA | 11 | D | 284 |
| 806220 | 152920 | 152935 | 3398 | 3413 | CCACAGTTCAGTCTAC | 6 | D | 285 |
| 806224 | 152967 | 152982 | 3445 | 3460 | CTCTTTGTAATCTAGA | 41 | D | 286 |
| 806228 | 153047 | 153062 | 3525 | 3540 | CATCAAGCCTGGACAA | 39 | D | 287 |
| 806232 | 153094 | 153109 | 3572 | 3587 | ACATAGGCATAAAAAT | 91 | D | 288 |
| 806236 | 153148 | 153163 | 3626 | 3641 | AAATATCATCTTGTTT | 54 | D | 289 |
| 806240 | 153192 | 153207 | 3670 | 3685 | TTAGATTAGGCAAGGA | 3 | D | 290 |
| 806244 | 153241 | 153256 | 3719 | 3734 | CAAAAATATACCATGT | 49 | D | 291 |
| 806248 | 153290 | 153305 | 3768 | 3783 | AATGAAGCTGTCTTCT | 35 | D | 292 |
| 806252 | 153340 | 153355 | 3818 | 3833 | GTGATTTAAGATTTAT | 32 | D | 293 |
| 806256 | 153366 | 153381 | 3844 | 3859 | TTTAATATTTATTACC | 102 | D | 294 |
| 806260 | 81835 | 81850 | 1294 | 1309 | CCATACTCTGCTGTGC | 9 | D | 295 |
| 806264 | 86382 | 86397 | 1346 | 1361 | TGGAAGTATCTTCATG | 20 | D | 296 |
| 806268 | 16840 | 16855 | N/A | N/A | AATGAATCATCTCTCT | 18 | D | 297 |
| 806272 | 30149 | 30164 | N/A | N/A | TGTCTCACAGACTCTA | 29 | D | 298 |
| | 91426 | 91441 | | | | | | |
| 806276 | 37417 | 37432 | N/A | N/A | AGGGATAATTTTCTAG | 40 | D | 299 |
| 806280 | 60193 | 60208 | N/A | N/A | GAGCATCTGATTCTGG | 37 | D | 300 |
| 806284 | 71093 | 71108 | N/A | N/A | AGCTATTGTGGAAGGT | 6 | D | 301 |
| | 97265 | 97280 | | | | | | |
| 806288 | 90247 | 90262 | N/A | N/A | AAAAGAGGGCCATAAC | 89 | D | 302 |
| 806292 | 95758 | 95773 | N/A | N/A | AAAATTCTTACTGGAA | 17 | D | 303 |
| | 96466 | 96481 | | | | | | |
| 806296 | 115933 | 115948 | N/A | N/A | GACATAACTATAGTGA | 20 | D | 304 |
| 806300 | 130196 | 130211 | N/A | N/A | TTATCTATATTATCTA | 87 | D | 305 |
| | 149100 | 149115 | | | | | | |
| 806304 | 151275 | 151290 | N/A | N/A | CAGGAAAAAATGTAAC | 79 | D | 306 |

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO: 3 (GENBANK Accession No. NM_015760.5).

TABLE 2
Reduction of NOX4 RNA by 3-10-3 cEt modified oligonucleotides with uniform phosphorothioate
internucleoside linkages at a concentration of 4000 nM in b.END cells

| | SEQ ID | SEQ ID | | | | |
|---|---|---|---|---|---|---|
| Compound | No: 3 | No: 3 | | NOX4 | | SEQ |
| Number | StartSite | Stop Site | Sequence (5′ to 3′) | (% UTC) | AID | ID NO |
| 806009 | 346 | 361 | CACAATCCTAGGCCCA | 67 | A | 307 |
| 806072 | 1259 | 1274 | ACACATTGTGAGAGTA | 24+ | D | 308 |

### Example 2: Dose-Dependent Reduction of Mouse NOX4 in b.END Cells by Modified Oligonucleotides

Modified oligonucleotides selected from the examples above were tested at various doses in b.END cells. Cultured b.END cells at a density of 20,000 cells per well were treated by electroporation with various concentrations of modified oligonucleotide as specified in the tables below. After a treatment period of approximately 24 hours, total RNA was isolated from the cells, and NOX4 RNA levels were measured by quantitative real-time RTPCR. Mouse NOX4 primer-probe set RTS35168 (described herein above) was used to measure RNA levels as described above. NOX4 RNA levels were normalized to total RNA content, as measured by RIBOGREEN®. Reduction of NOX4 RNA is presented in the tables below as percent NOX4 RNA, relative to the amount of NOX4 RNA in untreated control cells (% UTC).

The half maximal inhibitory concentration (IC₅₀) of each modified oligonucleotide was calculated using a linear regression on a log/linear plot of the data in Excel and is also presented in the tables below.

TABLE 3
Dose-dependent reduction of mouse NOX4 RNA
in b.END cells by modified oligonucleotides

| Compound | NOX4 RNA (% UTC) | | | | IC₅₀ |
|---|---|---|---|---|---|
| No. | 741 nM | 2222 nM | 6667 nM | 20000 nM | (μM) |
| 806013 | 39 | 24 | 7 | 3 | <0.7 |
| 806034 | 24 | 10 | 1 | 1 | <0.7 |
| 806070 | 33 | 12 | 5 | 1 | <0.7 |
| 806114 | 29 | 11 | 4 | 4 | <0.7 |
| 806130 | 18 | 5 | 3 | 2 | <0.7 |
| 806134 | 36 | 15 | 6 | 3 | <0.7 |
| 806154 | 16 | 6 | 3 | 3 | <0.7 |
| 806181 | 19 | 9 | 4 | 3 | <0.7 |
| 806194 | 28 | 10 | 6 | 1 | <0.7 |
| 806201 | 14 | 7 | 3 | 2 | <0.7 |
| 806202 | 21 | 8 | 4 | 2 | <0.7 |
| 806210 | 24 | 11 | 4 | 2 | <0.7 |
| 806221 | 19 | 9 | 4 | 2 | <0.7 |
| 806225 | 32 | 12 | 8 | 4 | <0.7 |
| 806240 | 21 | 7 | 3 | 1 | <0.7 |
| 806261 | 19 | 6 | 3 | 1 | <0.7 |
| 806277 | 18 | 5 | 4 | 3 | <0.7 |
| 806285 | 13 | 7 | 4 | 2 | <0.7 |

TABLE 4
Dose-dependent reduction of mouse NOX4 RNA
in b.END cells by modified oligonucleotides

| Compound | NOX4 RNA (% UTC) | | | | IC₅₀ |
|---|---|---|---|---|---|
| No. | 741 nM | 2222 nM | 6667 nM | 20000 nM | (μM) |
| 806036 | 22 | 14 | 5 | 3 | <0.7 |
| 806044 | 34 | 15 | 6 | 2 | <0.7 |
| 806052 | 41 | 15 | 4 | 1 | <0.7 |
| 806059 | 31 | 13 | 4 | 1 | <0.7 |
| 806063 | 29 | 15 | 2 | 2 | <0.7 |
| 806064 | 53 | 23 | 8 | 4 | <0.7 |
| 806110 | 62 | 40 | 30 | 17 | 1.44 |
| 806120 | 43 | 22 | 12 | 2 | <0.7 |
| 806131 | 36 | 17 | 9 | 2 | <0.7 |
| 806143 | 21 | 12 | 4 | 2 | <0.7 |
| 806163 | 32 | 15 | 5 | 3 | <0.7 |
| 806171 | 34 | 13 | 5 | 2 | <0.7 |
| 806179 | 26 | 10 | 5 | 3 | <0.7 |
| 806187 | 29 | 9 | 6 | 4 | <0.7 |
| 806212 | 14 | 6 | 2 | 1 | <0.7 |
| 806220 | 35 | 13 | 7 | 2 | <0.7 |
| 806240 | 25 | 5 | 2 | 1 | <0.7 |
| 806260 | 28 | 13 | 3 | 1 | <0.7 |
| 806267 | 24 | 6 | 2 | 1 | <0.7 |

### Example 3: Effect of a Modified Oligonucleotide Complementary to Mouse NOX4 in a Short-Term Cigarette Smoke Model of COPD

Two groups of eight, 10-week old male Balb/c mice (Taconic) were exposed to cigarette smoke (24 cigarettes per day) for a total of 4 continuous days (on Days 0, 1, 2 and 3). Another group of eight 10-week old male Balb/c mice (Taconic) were exposed to normal filtered air on Days 0, 1, 2, and 3.

One of the two groups of mice exposed to cigarette smoke received 3 loading doses Compound No. 806194 at 10 mg/kg on days −10, −7, and −3 by oropharyngeal aspiration. Following the loading doses, the mice were treated with Compound No. 806194 at 10 mg/kg on Days 0, and 2 by oropharyngeal aspiration.

The other group of mice exposed to cigarette smoke, and a group of mice that was not exposed to cigarette smoke (naïve) were treated with saline on days −10, −7, −3, 0, and 2 by oropharyngeal aspiration.

The mice were sacrificed on Day 4, forty-eight hours post the final administration of modified oligonucleotide. Treatment details are summarized in the table below. Lung function analysis, and BAL analysis was carried out on four of the eight mice from each group. RNA analysis was carried out on the remaining 4 animals from each group.

TABLE 5
Oropharyngeal aspiration treatment and smoking conditions
in short-term smoking COPD model study

| | Number | Compound dosed by | Smoking |
|---|---|---|---|
| Group | of mice | oropharyngeal aspiration | conditions |
| Naïve + | 8 | Saline [Days -10, -7, -3, | No smoke |
| Saline | | 0, and 2] | |
| Smoke + | 8 | Saline [Days -10, -7, -3, | Cigarette Smoke |
| Saline | | 0, and 2] | (24 cigarettes/day) |
| Smoke + | 8 | 806194 [Days -10, -7, -3, | Cigarette Smoke |
| 806194 | | 0, and 2 at 10 mg/kg/dose] | (24 cigarettes/day) |

### Lung Function Analysis

Lung function analysis was measured on Day 4 using the Penh score obtained through unrestrained plethysmography post methacholine challenge at 25 mg/ml using whole-body plethysmography (BuxcoElectronics, Sharon, CT). Airway hyperresponsiveness (AHR) was determined by inducing bronchoconstriction with methacholine aerosol at escalating doses (27). Total pulmonary airflow in unrestrained mice was estimated using a whole-body plethysmograph (Buxco Electronics, Sharon, CT). Pressure differences between a chamber containing an individual mouse and a reference chamber were used to extrapolate the enhanced pause (Penh). Penh is a dimensionless parameter that is a function of total pulmonary airflow in mice during each respiratory cycle. This parameter closely correlates with airway resistance as measured by traditional invasive techniques using ventilated mice. A higher Penh score indicates more lung constriction. The results, shown in the table below, indicate that pre-treatment with a modified oligonucleotide complementary to NOX4 prevented the increase in Penh score observed in the COPD mouse model.

TABLE 6
Lung Function (Penh) Scores

| Treatment | Penh Score |
|---|---|
| Naïve | 2 |
| Smoke | 3 |
| smoke + 806194 | 2 |

### Bronchoalveolar Lavage (BAL) Cellular Profile

Levels of macrophages (MAC), neutrophils (NEU), lymphocytes (LYM), and eosinophils (EOS) in the bronchoalveolar lavage fluid (BAL) were measured. Mouse lungs were lavaged two times with 0.5 ml of PBS containing 1% BSA (Sigma-Aldrich). BAL fluid samples were centrifuged to generate a cell pellet and a cell-free supernatant. The recovered airway cells were resuspended in PBS with 1% BSA, and a cytospin was performed. Cells were stained with Diff-Quik stain (VWR). Data are presented as the percent of cells present in the total recovered BAL cell population.

Extreme increases in neutrophils in BAL can be an indicator of acute and diffuse lung injury. Treatment with Compound No. 806194 resulted in a decrease in BAL neutrophil count.

TABLE 7
Cellular profile in BAL

| Treatment | MAC (%) | LYM (%) | EOS (%) | NEU (%) |
|---|---|---|---|---|
| Naïve | 97 | 2.3 | 0.0 | 1.0 |
| Smoke | 47 | 3.5 | 0.0 | 50.0 |
| Smoke + 806194 | 91 | 4.8 | 0.0 | 4.8 |

### RNA Analysis

On Day 4, RNA was extracted from the lungs of the mice for quantitative real-time RTPCR analysis of NOX4 RNA expression. The RTS35168 primer-probe set described above was used to measure levels of RNA of mouse NOX4.

RNA levels were normalized to mouse cyclophilin A levels. Mouse cyclophilin A was amplified using primer probe set m_cyclo24 (forward sequence TCGCCGCTTGCTGCA, designated herein as SEQ ID NO: 9; reverse sequence ATCGGCCGTGATGTCGA, designated herein as SEQ ID NO: 10; probe sequence CCATGGTCAACCCCACCGTGTTC, designated herein as SEQ ID NO: 11).

The levels ofNOX4 RNA expression are averaged for each group of mice and are presented as percent NOX4 RNA, relative to the amount in saline treated naïve animals, normalized to cyclophilin A (% control).

As presented in the table below, treatment with NOX4 modified oligonucleotide in the short-term smoking COPD model resulted in reduction of NOX4 RNA in comparison to the smoke+saline treated animals.

TABLE 8
Effect of modified oligonucleotides complementary
to mouse NOX4 on NOX4 RNA expression in a short-
term cigarette smoke-induced COPD model

| Treatment | % control NOX4 |
|---|---|
| Naïve + Saline | 100 |
| Smoke + Saline | 89 |
| Smoke + 806194 | 11 |

### Example 4: Effect of a Modified Oligonucleotide Complementary to Mouse NOX4 in the Sugen 5416/Hypoxia-Induced Mouse Model of Pulmonary Hypertension (PH)

Eleven-week-old male C₅₇BL/6J mice (Charles River Laboratories, Cambridge, MA) were given 3 loading doses of either saline or Compound No. 806194 at 10 mg/kg on days −10, −7, and −3 by oropharyngeal aspiration. Following the loading doses, the mice were treated with either saline or Compound No. 806194 on Days 0, 4, 7, 11, 14, 18, 21, and 25 (total 8 doses). In addition to treatment with saline or modified oligonucleotide, on Day 0, Day 7 and Day 14, the mice were injected subcutaneously with SU5416 (20 mg/kg) suspended with the aid of sonication in a mixture of 0.5% carboxymethylcellulose sodium (Sigma, St. Louis, MO), 0.9% sodium chloride (Sigma, St. Louis, MO), 0.4% polysorbate 80 (Sigma, St. Louis, MO), and 0.9% benzyl alcohol (Sigma, St. Louis, MO) in deionized water. On the same days (Day 0, Day 7 and Day 14), the mice were exposed to either room air (ambient oxygen levels) or chronic normobaric hypoxia inside a ventilated plexiglass chamber in which nitrogen was injected under the control of an Oxycycler controller (BioSpherix, Lacona, NY) to maintain hypoxia (10% oxygen). C₀₂ was monitored and ventilation was adjusted so that C₀₂ levels did not exceed 5,000 ppm (0.5%). Ammonia was removed by injection of pure air. After 3 weeks of exposure to 10% oxygen levels, and weekly injections of SU5416, animals were returned to normoxia for another week.

The mice were sacrificed on Day 28. Treatment details of each group of mice is specified in the table below.

TABLE 9
Disease establishment and treatment scheme in Sugen 5416/hypoxia-
induced mouse model of pulmonary hypertension

| | Number | Compound dosed by | |
|---|---|---|---|
| Group | of Mice | oropharyngeal aspiration | Oxygen Conditions |
| Saline | 8 | Saline (3 loading doses | Normal |
| | | followed by 8 doses on | |
| | | Days 0, 4, 7, 11, 14, | |
| | | 18, 21, and 25) | |
| Hypoxia + | 8 | Saline (3 loading doses | Hypoxic (Sugen |
| Saline | | followed by 8 doses on | 5416 + 10% oxygen |
| | | Days 0, 4, 7, 11, 14, | on (Day 0, Day 7 |
| | | 18, 21, and 25) | and Day 14) |
| Hypoxia + | 7 | 806194 (3 loading doses | Hypoxic (Sugen |
| 806194 | | at 10 mg/kg/dose followed | 5416 + 10% oxygen |
| | | by 8 doses on Days 0, 4, 7, | on (Day 0, Day 7 |
| | | 11, 14, 18, 21, and 25) | and Day 14) |

### RNA Analysis

On Day 28, RNA was extracted from the lungs of the mice for quantitative real-time RT-qPCR analysis of NOX4 RNA expression using primer-probe set RTS35168 (described herein above). RNA levels were normalized to mouse cyclophilin A levels, amplified using primer probe set m_cyclo24 (described herein above). The levels of NOX4 RNA expression are averaged for each group of mice and are presented as percent NOX4 RNA, relative to the amount in hypoxia+saline treated animals (% control).

As presented in the table below, treatment with NOX4 modified oligonucleotide in the Sugen 5416/hypoxia model of PH resulted in reduction of NOX4 RNA in comparison to the hypoxia+saline treated control.

TABLE 10
Effect of modified oligonucleotides complementary to mouse NOX4
on NOX4 RNA expression in a Sugen 5416/hypoxia-induced PH

| Treatment | % control NOX4 |
|---|---|
| Saline | 99 |
| Hypoxia + Saline | 100 |
| Hypoxia + 806194 | 15 |

### Heart Function

On Day 28, oxygen saturation was measured. Prior to euthanasia, oxygen saturation levels were measured using a MouseSTAT Pulse Oximeter (Kent Scientific).

On Day 28, right ventricle systolic pressure (RVSP), a PH phenotype, was measured in ventilated mice under 2.0%-2.5% isoflurane anesthesia delivered in room air with a flow of 2.0 L/min by inserting a 1.2 F mouse pressure catheter (Transonic Scisense Inc, ON, Canada) into the right ventricle via open chest method. Additionally, an indicator of RV (Right Ventricle) hypertrophy, the maximal rate of rise of right ventricular pressure (RV dP/dt_{MAX}) was measured during the RV catheterization as described herein above.

After euthanasia, the heart was isolated, and both atria were removed. The RV was carefully dissected from the LV (Left Ventricle) and the septum, and weighed (RV/LV+S).

As shown in the table below, treatment of the Sugen 5416/Hypoxia model with a modified oligonucleotide that targets NOX4 resulted in an improvement in RV/LV+S, oxygen saturation, RVSP, and RV dP/dt_{MAX}, compared to the saline-treated Sugen 5416/hypoxia control. Treatment resulted in a reduction of RV/LV+S, a reduction of RVSP, and a reduction of RV dP/dt_{MAX}, and an increase in oxygenation compared to the saline-treated Sugen 5416/hypoxia control.

TABLE 11
Effect of modified oligonucleotides complementary to mouse
NOX4 on heart function in a Sugen 5416/hypoxia-induced PH

| | | Oxygen | | |
|---|---|---|---|---|
| | RV/ | saturation | RVSP | dP/dt_{MAX} |
| Treatment | LV + S | (%) | (mmHg) | (mmHg/sec) |
| Saline | 0.28 | 97 | 22 | 1449 |
| Hypoxia + Saline | 0.43 | 85 | 32 | 2501 |
| Hypoxia + 806194 | 0.33 | 93 | 25 | 1623 |

### Example 5: Design of RNAi Agents Targeted to Mouse NOX4 Nucleic Acid

Double-stranded siRNA (siRNA) comprising antisense oligonucleotides complementary to mouse NOX4 nucleic acid, and sense oligonucleotides complementary to the antisense oligonucleotides are designed as follows.

The antisense oligonucleotide in each siRNA is 23 nucleosides in length; has a sugar motif (from 5′ to 3′) of: yfyfyfyfyfyfyfyfyfyfyyy; wherein each ‘y’ represents a ribo-2′-OMe sugar moiety, and each “f” represents a 2′-F sugar moiety; and an internucleoside linkage motif (from 5′ to 3′) of: ssooooooooooooooooooss; wherein ‘o’ represents a phosphodiester internucleoside linkage and ‘s’ represents a phosphorothioate internucleoside linkage. Each cytosine residue is a non-methylated cytosine. Each antisense oligonucleotide has a terminal phosphate at the 5′-end.

The sense oligonucleotide for each double-stranded siRNA is 21 nucleosides in length; has a sugar motif (from 5′ to 3′) of: fyfyfyfyfyfyfyfyfyfyf, wherein each ‘y’ represents a ribo-2′-OMe sugar moiety, and each “f” represents a 2′-F sugar moiety; and an internucleoside linkage motif (from 5′ to 3′) of: ssooooooooooooooooss; wherein ‘o’ represents a phosphodiester internucleoside linkage and ‘s’ represents a phosphorothioate internucleoside linkage. Each cytosine residue is a non-methylated cytosine.

Each antisense oligonucleotide is complementary to the target mouse NOX4 nucleic acid, which may be SEQ ID NO: 1 (GENBANK Accession No. NT_039433.8, truncated from nucleosides 4994000 to 5149000), or may be SEQ ID NO: 2 (GENBANK Accession No. NM_001285833.1), or may be both. Each antisense oligonucleotide may comprise at least 12, at least 13, at least 14, at least 15, or 16 contiguous nucleobases of the nucleobase sequence of any of SEQ ID NOs: 15-315.

Each sense oligonucleotide is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides).

Single-stranded RNAi (ssRNAi) RNAi agents comprising antisense oligonucleotides complementary to mouse NOX4 nucleic acid are designed as follows.

The antisense oligonucleotide in each siRNA is 23 nucleosides in length; has a sugar motif (from 5′ to 3′) of: yfyfyfyfyfyfyfyfyfyfyyy; wherein each ‘y’ represents a ribo-2′-OMe sugar moiety and each “f” represents a 2′-F sugar moiety; and an internucleoside linkage motif (from 5′ to 3′) of: ssooooooooooooooooooss; wherein ‘o’ represents a phosphodiester internucleoside linkage and ‘s’ represents a phosphorothioate internucleoside linkage. Each cytosine residue is a non-methylated cytosine. Each antisense oligonucleotide has a terminal phosphate at the 5′-end.

Each antisense oligonucleotide is complementary to the target mouse NOX4 nucleic acid, which may be SEQ ID NO: 1 (GENBANK Accession No. NT_039433.8, truncated from nucleosides 4994000 to 5149000), or may be SEQ ID NO: 2 (GENBANK Accession No. NM_001285833.1), or may be both. Each antisense oligonucleotide may comprise at least 12, at least 13, at least 14, at least 15, or 16 contiguous nucleobases of the nucleobase sequence of any of SEQ ID NOs: 12-308.

### Example 6: Effect of RNAi Agents Targeted to Mouse NOX4 Nucleic Acid on Mouse NOX4 RNA In Vitro, Single Dose

Cultured b.END cells are treated with RNAi agents designed according to Example 5 at a concentration of 0.1-20 nM by RNAiMAX at a density of 5,000 cells per well. After a treatment period of approximately 24 hours, total RNA is isolated from the cells and NOX4 RNA levels are measured by quantitative real-time RTPCR. NOX4 RNA levels are measured by mouse primer-probe set RTS35168 (described herein above). NOX4 RNA levels are normalized to total RNA content, as measured by RIBOGREEN®. Reduction of NOX4 RNA is assessed as percent NOX4 RNA relative to the amount of NOX4 RNA in untreated control cells (% UTC).

### Example 7: Effect of an RNAi Agent that Targets Mouse NOX4 in a Short-Term Cigarette-Smoke Model of COPD

Balb/c mice are either exposed to cigarette-smoke (24 cigarettes per day) for a total of 4 continuous days, or exposed similarly to normal filtered air. One group of mice exposed to smoke and one group of mice exposed to normal filtered air is treated with saline either by oropharyngeal aspiration or by aerosol administration. One group of mice exposed to smoke is treated with the RNAi agent either by oropharyngeal aspiration (at 10 mg/kg) or by aerosol administration (at 1 mg/kg).

### Lung Function Analysis

Lung function analysis is measured using the Penh score obtained through unrestrained plethysmography post methacholine challenge at 25 mg/ml using whole-body plethysmography (BuxcoElectronics, Sharon, CT). Airway hyperresponsiveness (AHR) is determined by inducing bronchoconstriction with methacholine aerosol at escalating doses (27). Total pulmonary airflow in unrestrained mice is estimated using a whole-body plethysmograph (Buxco Electronics, Sharon, CT). Pressure differences between a chamber containing an individual mouse and a reference chamber are used to extrapolate the enhanced pause (Penh). Penh is a dimensionless parameter that is a function of total pulmonary airflow in mice during each respiratory cycle. This parameter closely correlates with airway resistance as measured by traditional invasive techniques using ventilated mice. A higher Penh score indicates more lung constriction.

Treatment with an RNAi agent results in a decrease in the Penh score compared to saline treated animals in the short-term cigarette-smoke model of COPD.

### Bronchoalveolar Lavage (BAL) Cellular Profile

Levels of macrophages, neutrophils, lymphocytes, and eosinophils in the bronchoalveolar lavage fluid (BAL) are measured. Mouse lungs are lavaged two times with 0.5 ml of PBS containing 1% BSA (Sigma-Aldrich). BAL fluid samples are centrifuged to generate a cell pellet and a cell-free supernatant. The recovered airway cells are resuspended in PBS with 1% BSA, and a cytospin is performed. Cells are stained with Diff-Quik stain (VWR). Data are presented as the percent of cells present in the total recovered BAL cell population.

Extreme increases in neutrophils in BAL can be an indicator of acute and diffuse lung injury. Treatment with an RNAi agent results in a decrease in BAL neutrophil count in the short-term cigarette-smoke model of COPD.

### RNA Analysis

RNA is extracted from the lungs of the mice for quantitative real-time RT-PCR analysis of NOX4 RNA expression. The RTS35168 primer-probe set described above is used to measure levels of RNA of mouse NOX4. RNA levels are normalized to mouse cyclophilin A levels, amplified using primer probe set m_cyclo24 (described herein above). The levels of NOX4 RNA expression are averaged for each group of mice and are assessed as percent NOX4 RNA, relative to the amount in saline treated naïve animals, normalized to cyclophilin A (% control).

Treatment with an RNAi agent that targets NOX4 in the short-term smoking COPD model results in reduction of NOX4 RNA in comparison to the smoke+saline treated animals.

### Example 8: Effect of RNAi Agents that Target Mouse NOX4 in the Sugen 5416/Hypoxia-Induced Mouse Model of Pulmonary Hypertension (PH)

Hypoxic conditions are induced by subcutaneous injection with SU5416 (20 mg/kg) suspended with the aid of sonication in a mixture of 0.5% carboxymethylcellulose sodium (Sigma, St. Louis, MO), 0.9% sodium chloride (Sigma, St. Louis, MO), 0.4% polysorbate 80 (Sigma, St. Louis, MO), and 0.9% benzyl alcohol (Sigma, St. Louis, MO) in deionized water. On the same day as the SU5416 treatment, the mice are exposed to either room air (ambient oxygen levels) or chronic normobaric hypoxia inside a ventilated plexiglass chamber in which nitrogen is injected under the control of an Oxycycler controller (BioSpherix, Lacona, NY) to maintain hypoxia (10% oxygen). CO₂ is monitored and ventilation is adjusted so that CO₂ levels will not exceed 5,000 ppm (0.5%). Ammonia is removed by injection of pure air. After 3 weeks of exposure to 10% oxygen levels, and weekly injections of SU5416, animals are returned to normoxia for another week before they are sacrificed

One group of mice exposed to hypoxic conditions and one group of mice exposed to normal air are treated with saline either by oropharyngeal aspiration or by aerosol administration. One group of mice subjected to hypoxic conditions is treated with the RNAi agent either by oropharyngeal aspiration (at 10 mg/kg) or by aerosol administration (at 1 mg/kg).

### RNA Analysis

RNA is extracted from the lungs of the mice for quantitative real-time RT-qPCR analysis of NOX4 RNA expression. The RTS35168 primer-probe set described above is used to measure levels of RNA of mouse NOX4. RNA levels are normalized to mouse cyclophilin A levels, amplified using primer probe set m_cyclo24 (described herein above). The levels of NOX4 RNA expression are averaged for each group of mice and are assessed as percent NOX4 RNA, relative to the amount in hypoxia+saline treated animals (% control).

Treatment with RNAi agents targeted to NOX4 in the Sugen 5416/hypoxia model of PH results in reduction of NOX4 RNA in comparison to the hypoxia+saline treated control.

### Heart Function

Prior to euthanasia, oxygen saturation levels are measured using a MouseSTAT Pulse Oximeter (Kent Scientific). Oxygen saturation improves in the Sugen 5416/hypoxia model of PH after treatment with an RNAi agent that targets mouse NOX4 relative to the Sugen 5416/hypoxia control animals.

Right ventricle systolic pressure (RVSP), a PH phenotype, is measured in ventilated mice under 2.0%-2.5% isoflurane anesthesia delivered in room air with a flow of 2.0 L/min by inserting a 1.2 F mouse pressure catheter (Transonic Scisense Inc, ON, Canada) into the right ventricle via open chest method. RVSP increases in the Sugen 5416/hypoxia model of PH compared to naïve animals. RVSP is found to be reduced in the Sugen 5416/hypoxia model of PH after treatment with an RNAi agent that targets mouse NOX4 relative to the Sugen 5416/hypoxia control animals.

Additionally, an indicator of RV (Right Ventricle) hypertrophy, RV dP/dt_{MAX} is measured during RV catheterization as described herein above. RV hypertrophy is an abnormal enlargement or pathologic increase in muscle mass of the right ventricle in response to pressure overload. RV hypertrophy is found to be reduced in the Sugen 5416/hypoxia model of PH after treatment with an RNAi agent that targets mouse NOX4 relative to the Sugen 5416/hypoxia control animals.

The extent of right ventricular hypertrophy is estimated by calculation of the ratio of RV/LV+S. A higher RV/LV-S ratio is associated with increased levels of right ventricular hypertrophy. After euthanasia, the heart is isolated, and both atria are removed. The RV is carefully dissected from the LV (Left Ventricle) and the septum (S), and weighed to determine the (RV/LV+S) ratio. The RV/LV+S ratio is found to be reduced in the Sugen 5416/hypoxia model of PH after treatment with an RNAi agent that targets mouse NOX4 relative to the Sugen 5416/hypoxia control animals.

### Example 9: Effect of Modified Oligonucleotides or RNAi Agents that Target Rat NOX4 in a Rat Sugen 5416/Hypoxia Model of Pulmonary Hypertension (PH)

Sprague-Dawley rats are dosed subcutaneously with 30 mg/kg Sugen 5416 and exposed to 21 days of hypobaric hypoxia. Rats are then maintained in normoxia for 21 days. A separate group of rats are exposed to normal air.

One group of rats exposed to hypoxic conditions and one group of rats exposed to normal air are treated with saline either by oropharyngeal aspiration or by aerosol administration. One group of rats subjected to hypoxic conditions is treated with the modified oligonucleotide or the RNAi agent either by oropharyngeal aspiration (at ˜1-3 mg/kg) or by aerosol administration (at ˜0.3-1 mg/kg).

Treatment with modified oligonucleotides or RNAi agents targeted to NOX4 in the rat Sugen 5416/hypoxia model of PH results in reduction of NOX4 RNA in comparison to the hypoxia+saline treated control.

### Heart Function

Prior to euthanasia, oxygen saturation levels are measured using a pulse oximeter. Oxygen saturation improves in the Sugen 5416/hypoxia model of PH after treatment with a modified oligonucleotide or an RNAi agent that targets rat NOX4 relative to the Sugen 5416/hypoxia control animals.

Right ventricle systolic pressure (RVSP), a PH phenotype, is measured in ventilated rats under 2.0%-2.5% isoflurane anesthesia delivered in room air by inserting a catheter into the right ventricle via open chest method. RVSP increases in the Sugen 5416/hypoxia model of PH compared to naïve animals. RVSP is found to be reduced in the Sugen 5416/hypoxia model of PH after treatment with a modified oligonucleotide or an RNAi agent that targets rat NOX4 relative to the Sugen 5416/hypoxia control animals.

Additionally, an indicator of RV (Right Ventricle) hypertrophy, RV dP/dtAx is measured during RV catheterization as described herein above. RV hypertrophy is an abnormal enlargenent or pathologic increase in muscle mass of the right ventricle in response to pressure overload. RV hypertrophy is found to be reduced in the Sugen 5416/hypoxia model of PH after treatment with a modified oligonucleotide or an RNAi agent that targets rat NOX4 relative to the Sugen 5416/hypoxia control animals.

The extent of right ventricular hypertrophy is estimated by calculation of the ratio of RV/LV-+S. A higher RV/LV+S ratio is associated with increased levels of right ventricular hypertrophy. After euthanasia, the heart is isolated, and both atria are removed. The RV is carefully dissected from the LV (Left Ventricle) and the septum (S), and weighed to determine the (RV/LV+S) ratio. The RV/LV+S ratio is found to be reduced in the Sugen 5416/hypoxia model of PH after treatment with a modified oligonucleotide or an RNAi agent that targets rat NOX4 relative to the Sugen 5416/hypoxia control animals.

What is claimed is:

1. A method of treating a pulmonary disease or disorder in a subject having, or at risk of having, a pulmonary disease or disorder comprising administering a NOX4-specific inhibitor to the subject, thereby treating the pulmonary disease or disorder in the subject.

2. The method of claim 1, wherein the pulmonary disease or disorder is chronic obstructive pulmonary disease (COPD) or pulmonary hypertension (PH).

3. The method of claim 1 or claim 2, wherein the administration of the NOX4-specific inhibitor ameliorates at least one symptom of the pulmonary disease or disorder.

4. The method of claim 3, wherein the symptom is coughing, wheezing, difficulty breathing, shortness of breath, chest pressure, chest pain, edema in one or both ankles, edema in one or both legs, edema in one or both feet, vascular remodeling, low blood oxygen, increased blood pressure, fatigue, dizziness, frequent respiratory infections, or coughing with mucus.

5. The method of any of claims 1-4, wherein administration of the NOX4-specific inhibitor reduces coughing, reduces wheezing, improves breathing, reduces shortness of breath, reduces chest pressure, reduces chest pain, reduces edema in one or both ankles, reduces edema in one or both legs, reduces edema in one or both feet, reduces or reverses vascular remodeling, increases blood oxygen, decreases blood pressure, reduces fatigue, reduces dizziness, reduces the frequency of respiratory infections, reduces coughing with mucus, or a combination thereof.

6. The method of any of claims 1-5, wherein administering the NOX4-specific inhibitor improves spirometry, lung function, oxygen saturation, or blood pressure, or a combination thereof.

7. The method of any of claims 1-6, wherein administering the NOX4-specific inhibitor reduces bronchial tube inflammation, increases spirometry levels of forced vital capacity, increases spirometry levels of forced expiratory volume, improves lung function, increases oxygen saturation, or decreases blood pressure, or a combination thereof.

8. The method of any of claims 1-7, wherein the subject is a human subject.

9. A method of inhibiting expression or activity of NOX4 in a cell comprising contacting the cell with a NOX4-specific inhibitor, thereby inhibiting expression or activity of NOX4 in the cell.

10. The method of claim 9, wherein the cell is a human cell.

11. The method of claim 9 or claim 10, wherein the cell is a lung cell.

12. The method of any of claims 9-11, wherein the cell is in a subject.

13. The method of claim 12, wherein the subject is human.

14. The method of claim 12 or claim 13, wherein the subject has, or is at risk of having a pulmonary disease or disorder.

15. The method of claim 14, wherein the pulmonary disease or disorder is COPD, or PH.

16. Use of a NOX4-specific inhibitor for the manufacture or preparation of a medicament for treating a pulmonary disease or disorder.

17. Use of a NOX4-specific inhibitor for the treatment of a pulmonary disease or disorder.

18. The use of claim 16 or claim 17, wherein the pulmonary disease or disorder is COPD or PH.

19. The use of any of claims 16-18, wherein the NOX4-specific inhibitor ameliorates at least one symptom of the pulmonary disease or disorder.

20. The use of claim 19, wherein the symptom is coughing, wheezing, difficulty breathing, shortness of breath, chest pressure, chest pain, edema in one or both ankles, edema in one or both legs, edema in one or both feet, vascular remodeling, low blood oxygen, increased blood pressure, fatigue, dizziness, frequent respiratory infections, or coughing with mucus

21. The use of claim 19 or claim 20, wherein the NOX4-specific inhibitor reduces coughing, reduces wheezing, improves breathing, reduces shortness of breath, reduces chest pressure, reduces chest pain, reduces edema in one or both ankles, reduces edema in one or both legs, reduces edema in one or both feet, reduces or reverses vascular remodeling, increases blood oxygen, decreases blood pressure, reduces fatigue, reduces dizziness, reduces the frequency of respiratory infections, reduces coughing with mucus, or a combination thereof.

22. The use of any of claims 16-21, wherein the NOX4-specific inhibitor improves spirometry, lung function, oxygen saturation, or blood pressure, or a combination thereof.

23. The use of any of claims 16-22, wherein the NOX4-specific inhibitor reduces bronchial tube inflammation, increases spirometry levels of forced vital capacity, increases spirometry levels of forced expiratory volume, improves lung function, increases oxygen saturation, or decreases blood pressure, or a combination thereof.

24. The method or use of any of claims 1-23, wherein the NOX4-specific inhibitor is an antisense agent, a polypeptide, an antibody, or a small molecule.

25. The method or use of any of claims 1-24, wherein the NOX4-specific inhibitor is an antisense agent comprising a modified oligonucleotide, wherein the modified oligonucleotide has a nucleobase sequence complementary to any one of SEQ ID NOs: 1-5.

26. The method or use of claim 25, wherein the nucleobase sequence of the modified oligonucleotide is complementary to SEQ ID NO: 3 or SEQ ID NO: 4.

27. The method or use of claim 26, wherein the nucleobase sequence of the modified oligonucleotide is at least 90% complementary to the nucleobase sequence of an equal length portion of SEQ ID NO: 3 or SEQ ID NO: 4.

28. The method or use of claim 26, wherein the nucleobase sequence of the modified oligonucleotide is at least 95% complementary to the nucleobase sequence of an equal length portion of SEQ ID NO: 3 or SEQ ID NO: 4.

29. The method or use of claim 28, wherein the nucleobase sequence of the modified oligonucleotide is 100% complementary to the nucleobase sequence of an equal length portion of SEQ ID NO: 3 or SEQ ID NO: 4.

30. The method or use of any of claims 24-29, wherein the antisense agent is single-stranded.

31. The method or use of any of claims 24-29, wherein the antisense agent is double-stranded.

32. The method or use of any of claims 25-29, wherein the modified oligonucleotide consists of 12 to 30 linked nucleosides.

33. The method or use of any of claims 25-32, wherein at least nucleoside of the modified oligonucleotide comprises a modified nucleobase.

34. The method or use of claim 33, wherein the modified nucleobase is a 5-methylcytosine.

35. The method or use of any of claims 25-34, wherein at least one nucleoside of the modified oligonucleotide comprises a modified sugar moiety.

36. The method or use of claim 35, wherein the modified sugar moiety is a bicyclic sugar moiety.

37. The method or use of claim 36, wherein the modified sugar comprises a 4′-CH(CH₃)—O—2′ bridge or a 4′-(CH₂)ₙ—O—2′ bridge, wherein n is 1 or 2.

38. The method or use of claim 37, wherein the modified sugar moiety comprises a non-bicyclic modified sugar moiety.

39. The method or use of claim 38, wherein the non-bicyclic sugar moiety is a 2′-F, 2′-OMe, or 2′-MOE sugar moiety.

40. The method or use of any of claims 25-39, wherein the modified oligonucleotide has:
a gap segment consisting of linked 2′-deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar moiety.

41. The method or use of any of claims 25-39, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprise a 2′-substituted sugar moiety.

42. The method or use of claim 41, wherein the central region consists of 6-10 nucleosides comprising a 2′-β-D-deoxyribosyl sugar moiety.

43. The method or use of any one of claims 25-42, wherein at least one internucleoside linkage of the modified oligonucleotide is a modified internucleoside linkage.

44. The method or use of claim 43, wherein the at least one modified internucleoside linkage is a phosphorothioate internucleoside linkage.

45. The method or use of claim 44, wherein each internucleoside linkage is a phosphorothioate internucleoside linkage.

46. The method or use of claim 44, wherein each internucleoside linkage is independently selected from a phosphodiester internucleoside linkage and a phosphorothioate internucleoside linkage.

47. The method or use of any of claims 25-46, wherein the antisense agent comprises a conjugate group.

48. The method or use of any of claims 1-47, wherein the NOX4-specific inhibitor is an RNase H agent capable of reducing the amount of NOX4 nucleic acid through the activation of RNase H.

49. The method or use of any of claims 1-47, wherein the NOX4-specific inhibitor is an RNAi agent capable of reducing the amount of NOX4 nucleic acid through the activation of RISC/Ago2.

50. The method or use of any of claims 1-47, wherein the NOX4-specific inhibitor is a steric-blocking agent capable of directly binding to a target nucleic acid, thereby blocking the interaction of the NOX4 nucleic acid with other nucleic acids or proteins.

51. The method of any of claims 1-8 or 16-50, wherein a therapeutic amount of the NOX4-specific inhibitor is administered to the subject.

52. The method of any of claims 1-8 or 16-51, wherein the NOX4-specific inhibitor is administered by aerosolized delivery.

53. The method of any of claims 1-8 or 16-51, wherein the NOX4-specific inhibitor is administered via a nebulizer or inhaler.
